(19) United States
(12) Patent Application Publication
(10) Pub. No.: US 2007/0092585 A1
(43) Pub. Date: Apr. 26, 2007

(54) Cancer chemotherapy compositions comprising PI3K pathways modulators and triptolide

(76) Inventor: MIchael K. Skinner, Pullman, WA (US)

Correspondence Address: MANDEL & ADRIANO, 55 SOUTH LAKE AVENUE, SUITE 710, PASADENA, CA, 91101, US

(21) Appl. No.: 11/545,909

(22) Filed: Oct. 11, 2006

Related U.S. Application Data
(60) Provisional application No. 60/726,969, filed on Oct. 14, 2005.

(51) Int. Cl. A61K 31/365; A61K 31/366; A61K 31/282; A61K 31/337; A61K 33/24

(52) U.S. Cl. 424/649; 514/454; 514/449; 514/492; 514/468

(57) ABSTRACT

The present invention provides compositions and methods for inhibiting growth of and/or killing cancer cells. The compositions include: an inhibitor of the PI3K signal transduction pathway, and additional agents, such as Triptolide.

This application claims priority to provisional patent application, U.S. Ser. No. 60/726,969, filed Oct. 14, 2005, the contents of which are hereby incorporated by reference in their entirety herein.

Throughout this application various publications are referenced. The disclosures of these publications in their entireties are hereby incorporated by reference into this application in order to more fully describe the state of the art to which this invention pertains.

### FIELD OF INVENTION

The present invention relates generally to compositions and methods for inhibiting growth of or killing cancer cells. In particular, the invention is directed toward inhibiting growth and/or killing cancer cells by modulating the PI3K signal transduction pathway, and using additional therapeutic agents, such as triptolide. The present invention is also directed toward growth inhibition or killing of cancer cells, using the compositions and methods of the present invention, in combination with traditional chemotherapeutic agents, and/or radiation therapy.

### BACKGROUND OF THE INVENTION

Signal transduction pathways that mediate cell growth and survival are targets for cancer therapy, as tumorigenesis often involves dysfunctional signal transduction pathways. Signaling abnormalities provide cancer cells increased growth potential, and the ability to avert apoptosis induced by DNA damaging agents.

In particular, the PI3K signal transduction pathway is known to mediate normal and abnormal processes that lead to tumorigenesis or tumor metastasis. Standard treatments currently used for various solid tumors include surgery, radiotherapy, chemotherapy, and/or hormone therapy. Ionizing radiation therapy is generally the therapy of choice for the treatment of many cancers. However, it is well known that incomplete killing of neoplastic cells can result in the recurrence of cancer even after rigorous radiation treatment regimens are completed. Furthermore, some cell populations are stimulated to proliferate as a result of exposure to radiation. The molecular mechanism(s) by which tumor cells are killed, survive or stimulated to proliferate after exposure to ionizing radiation are not fully understood. Several reports have demonstrated that radiation activates multiple signaling pathways within cells in vitro which can lead to either increased cell death or increased proliferation depending upon the dose and culture conditions (Xia, Dickens et al. 1995; Kasid, Suy et al. 1996; Kyriakis and Avruch 1996; Rosette and Karin 1996; Santana, Pena et al. 1996; Verheij, Bose et al. 1996; Chmura, Nodzenski et al. 1997).

However, some tumors are unresponsive or become refractive to these therapies, or worse, some tumors are stimulated to proliferate by radiotherapy.

Management of various solid tumors, including ovarian, testicular, head and neck, bladder and lung cancer remains a challenge. For example, ovarian cancer ranks fifth as a cause of cancer deaths among women, and results in more deaths than any other cancer of the female reproductive system (Piver, Baker et al. 1991).

Newer treatment regimens involving platinum-containing drugs and paclitaxel, have improved survival length, but have significant adverse side effects, and are not effective for managing recurrent or persistent ovarian cancer. Most patients who are initially responsive to this therapy, eventually become resistant. Therefore, drug resistance remains one of the largest obstacles in the treatment of patients with recurrent disease.

Chemotherapeutic agents currently in use for treatment of ovarian cancer include platinum-containing compounds such as cisplatin, carboplatin and oxaliplatin (Council 2000; Misset, Bleiberg et al. 2000; Thigpen 2000) and transplatin. These agents are used alone, or more commonly in combination with cyclophosphamide, or taxol or its analogue, paclitaxel (Council 2000; Markman 2000; Misset, Bleiberg et al. 2000; Ozols 2000; Thigpen 2000).

Cisplatin kills cells by inducing DNA damage (J Reedijk 1987 Pure Appl Chem 59:181-192). However, DNA damage can sometimes lead to cell stress responses resulting in cytotoxicity or survival via activation of various signal transduction pathways to induce cell cycle arrest, DNA repair, survival or apoptosis. Thus, cisplatin treatment of certain cancers sometimes leads to drug-resistant tumors (Eastman 1990; Ozols 1992; Ferguson 1995; Gosland, Lum et al. 1996; Crul, Schellens et al. 1997; Nehme, Baskaran et al. 1997).

Previous studies have shown that inhibiting the PI3K pathway can sensitize ovarian cancer cell lines to the killing effects of platinum-containing drugs such as cisplatin. Thus agents and methods that inhibit the PI3K pathway may offer new treatment regimens for certain cancers.

The PI3K signal transduction pathway is a phosphatidylinositol 3-kinase pathway which mediates and regulates cellular apoptosis (Yao and Cooper 1995; Minshall, Arkins et al. 1996; Yao and Cooper 1996). The PI3K pathway also mediates cellular processes, including proliferation, growth, differentiation, motility, neovascularization, mitogenesis, transformation, viability, and senescence (Carpenter, Duckworth et al. 1990; Pignataro and Ascoli 1990; Varticovski, Harrison-Findik et al. 1994; Hu, Klippel et al. 1995; Grasso, Wen et al. 1997).The cellular factors that mediate the PI3K pathway include PI3K, Akt, and BAD, that mediate and regulate cellular apoptosis (Yao and Cooper 1995; Minshall, Arkins et al. 1996; Yao and Cooper 1996). The PI3K factors include class I PI3K, a cytostolic enzyme complex which includes p85 and p110 (Carpenter and Cantley 1990). PI3K plays a role in cellular proliferation, motility, neovascularization, viability, and senescence (Carpenter, Duckworth et al. 1990; Varticovski, Harrison-Findik et al. 1994; Hu, Klippel et al. 1995). PI3K is also mediates cellular differentiation of certain cell types (Pignataro and Ascoli 1990; Grasso, Wen et al. 1997).Akt is a threonine/serine kinase (Dudek, Datta et al. 1997; Kauffinann-Zeh, Rodriguez-Viciana et al. 1997; Kennedy, Wagner et al. 1997; Khwaja, Rodriguez-Viciana et al. 1997; Kulik, Klippel et al. 1997).BAD has been identified as a pro-apoptotic member of the bcl-2 family (Datta, Dudek et al. 1997; del Peso, Gonzalez-Garcia et al. 1997).

In particular, cellular factors that mediate the PI3K pathway have been implicated in certain cancers. For example, gene amplification of the P110-alpha subunit of PI3K occurs in approximately 80% of primary ovarian cancer cells (Shayesteh, Lu et al. 1999).

Agents, such as LY294002 and Wortmannin have been previously shown to inhibit the activity of cellular factors that mediate the PI3K pathway. The agent LY294002 has the chemical formula (2-(4-morpholinyl)-8-phenyl-4H-1-benzopyran-4-one) which is an analog of quercetin, a naturally-occurring bioflavinoid. LY294002 has been shown to specifically inhibit PI3K activity by binding at the ATP-binding site of PI3K (Vlahos, Matter et al. 1994). It is also a competitive, reversible inhibitor of the ATP binding site of PI3K (Vlahos, Matter et al. 1994). LY294002 also inhibits Retinoblastoma protein hyperphosphorylation that normally occurs during G1 cell cycle progression and induces an increase in activity of p27, a cyclin-dependent kinase inhibitor (Casagrande, Bacqueville et al. 1998). LY294002 has been shown to inhibit melanoma cell proliferation (Casagrande, Bacqueville et al. 1998), and partial inhibition of osteosarcoma proliferation (Thomas, Venugopalan et al. 1997).

LY294002 has been used to inhibit expression of p21 but had no effect on expression of the pro-apoptotic protein BAX, in the presence of cisplatin or paclitaxel in cultured ovarian cancer cells (Mitsuuchi, Johnson et al. 2000). LY294002 has also been used to reduce tumor burden in an ovarian cancer mouse model (Hu, Zaloudek et al. 2000).

The agent Wortmannin has the chemical formula [1S-(1α,6bα,9aβ,11bβ)]-11-(Acetyloxy)-1, 6b,7,8,9a,10,11,11b-octahydro-1(methoxymethyl)-9a,11b-dimethyl-3H-furo [4,3,2-de]indeno[4,5-h]-2-benzopyran-3,6,9-trione. Wortmannin is an antifungal antibiotic isolated from various species of Penicillium and has been previously shown to be a selective inhibitor of phosphatidylinositol 3-kinase (Arcaro and Wymann 1993; Ridderstrale and Tornqvist 1994).

The agents LY294002 or Wortmannin effectively blocked fibronectin-dependent (FN) secretion of matrix metalloproteinase (MMP-9) in an ovarian cancer cell line (Thant, Nawa et al. 2000).

In one study, PD098059 (MAPK inhibitor) and Wortmannin (PI13K inhibitor) were used in combination to sensitize cultured ovarian cancer cells to the cytotoxic effects of cisplatin (Hayakawa, Ohmichi et al. 2000).

Another agent that exerts anti-tumor activity is Triptolide (TPL), a diterpenoid triexposide purified from a Chinese herb Tripterygium Wilfordii Hook F (or the acronym: TWHF). TWHF has been used in traditional Chinese medicine for thousands of years. Triptolide is an effective immunosuppressive and anti-inflammatory agent, and has been used for the treatment of autoimmune diseases, especially rheumatoid arthritis (Chen 2001). It inhibits both calcium-dependent and calcium-independent pathways. It also affects T cell activation by inhibiting interleukin-2 transcription. Triptolide also inhibits expression of pro-inflammatory cytokines (Lin, Sato et al. 2001) and inhibits expression of adhesion molecules produced by epithelial cells.

It was later discovered that in addition to immunosuppressive activities, TPL possesses anti-inflammatory and antifertility activities (Lue, Sinha Hikim et al. 1998; Qiu and Kao 2003). Moreover, it was recently discovered that TPL has potent anti-neoplastic and anti-tumor activities (Shamon, Pezzuto et al. 1997; Lee, Chang et al. 1999; Jiang, Wong et al. 2001). Despite the multi-functional nature of this drug, the exact molecular mechanisms of its actions are largely unknown.

Antiproliferative and pro-apoptotic activities of TPL have been shown with several different types of cancer cells in vitro and in vivo. TPL has been shown to induce apoptosis in leukemia cells in vitro (Yang, Liu et al. 1998; Chan, Cheng et al. 2001) and to have significant antagonistic activity against mouse leukemia in vivo (Zhang, Chen et al. 1981). Clinical trials in China have demonstrated that TPL treatment can induce a high remission rate in both mononuclocytic and granulocytic leukemias (Lu, Lian et al. 1992). TPL was also found to be effective in inhibiting proliferation of human gastric cancer cells and prostatic epithelial cells in vitro (Jiang, Wong et al. 2001; Kiviharju, Lecane et al. 2002). TPL can inhibit the growth of several different solid tumor types including breast, bladder, stomach and melanomas (Yang, Chen et al. 2003). TPL is able to inhibit the transcriptional activities of the tumor suppressor p53, and the nuclear factor κB (NFκB). As both p53 and NFκB have been implicated in the progression and chemoresistance of ovarian cancer, TPL is a potential chemotherapeutic for ovarian cancer.

TPL was also found to be more effective on a molar basis than both cisplatin and taxol in inhibiting xenograft growth of these tumor types (Yang, Chen et al. 2003). TPL also potentiates the activities of other agents (Chang, Kang et al. 2001; Lee, Park et al. 2002; Fidler, Li et al. 2003).

The platinum-based compounds, cisplatin and carboplatin, bind to DNA and nuclear proteins leading to the formation of DNA crosslinks that inhibit DNA replication and transcription (Herrin and Thigpen 1999; Shepherd 2000). In ovarian cancer the chemoresistance that develops to these DNA damaging agents is thought to be largely mediated through p53-induced cell cycle arrest (Ferreira, Tolis et al. 1999; Vasey 2003; Blumenthal, Leone et al. 2004). TPL has been shown to enhance doxorubicin-mediated apoptosis of tumor cells by blocking p53-mediated cell cycle arrest (Chang, Kang et al. 2001). In addition to p53, intrinsically or constitutively activated NFκB is thought to confer drug resistance in several cancer types, including ovarian cancer (Baldwin 2001). A recent study demonstrated that inhibition of NFκB activity resulted in increased efficacy of cisplatin in ovarian cancer cells (Mabuchi, Ohmichi et al. 2004). Several studies have shown that TPL is able to inhibit the transcriptional activity of NFκB (Jiang, Wong et al. 2001; Lee, Park et al. 2002; Qiu and Kao 2003). Therefore TPL may be effective in attenuating chemoresistance in ovarian cancer cells through inhibition of NFκB and/or p53 activities.

The anti-tumor effects of compositions can be monitored in an animal model for ovarian cancer. An animal model harboring an ovarian carcinoma cell line A2780 which transiently expresses an SEAP protein as a reporter gene has previously been reported (Bao, Selvakumaran et al. 2000). Bao found that the serum levels of the SEAP protein found in the animal correlated with tumor burden in response to paclitaxel.

A nude mouse tumor model has been developed, that has been shown to be effective in evaluating the response of ovarian tumor xenografts in nude mice (Nilsson, Westfall et al. 2002). This in vivo tumor model involves a human ovarian cancer cell line, OCC1, which has been stably transfected with a secreted alkaline phosphatase (SEAP) gene. The SEAP reporter gene is constitutively expressed, and has been shown to be secreted at levels proportional to the number of tumor cells present (Nilsson, Westfall et al. 2002). Thus, this mouse model can be used to evaluate the in vivo effects of various agents for treating cancer.

Thus, there remains a need for new compositions and methods, for inhibiting and/or killing tumor cells, and for improving existing cancer treatment methods, via therapeutic compounds, and modulators of signal transduction pathways, such as the PI3K pathway.

### SUMMARY OF THE INVENTION

Accordingly, the present invention provides compositions and methods that are useful for treating disease or cancer, caused by a dysfunctional signal transduction pathway, such as the PI3K pathway, agents that modulate the pathway, in combination with additional compounds such as triptolide, and chemotherapeutic agents.

The compositions of the invention include compounds that modulate the PI3K signal transduction pathways, thereby inhibiting growth of, or killing cancer cells.

In one embodiment, the compositions of the invention comprise an agent that inhibits the PI3K signal transduction pathway, in combination with triptolide.

In another embodiment, the compositions of the invention comprise adjunct chemotherapy agents such as carboplatin, and an agent that inhibits the PI3K signal transduction pathway, in combination with triptolide, for treating cancer.

The present invention further provides the use of an ovarian cancer animal model harboring an ovarian carcinoma cell line OCC1, which is stably transfected to express SEAP in the methods of detecting the anti-tumor effects of the compositions of the invention.

### BRIEF DESCRIPTION OF FIGURES

FIG. 1: A graph showing correlation between calculated subcutaneous tumor volume and plasma SEAP levels, in nude mice injected with OCC1-SEAP cells, as described in Example I, infra.

FIG. 2: Graphs showing plasma SEAP levels and tumor volume in invasive tumors in nude mice, as described in Example I, infra. (A) Tumor diameter, measured through the skin and tumor volume calculated. (B) Plasma SEAP levels from blood samples.

FIG. 3: A graph showing correlation between intraperitoneal tumor mass and plasma SEAP levels in nude mice, as described in Example I, infra.

FIG. 4: Graphs showing correlation between cell density and secreted SEAP levels in vitro, as described in Example I, infra. (A) DNA levels were determined to reflect cell numbers in each well. (B) SEAP levels were determined in the medium of cultured cells at 24 hours. (C) SEAP secretion from OCC1-SEAP cells was normalized per microgram DNA in each well.

FIG. 5: Graphs showing the response of OCC1-SEAP cells to carboplatin chemotherapy in vitro, as described in Example I, infra. (A) The amount of DNA per well was measured in control and carboplatin-treated cultures. (B) SEAP levels were assayed in the medium of cultured cells after the treatment period. (C) SEAP secretion from OCC1-SEAP cells was normalized per microgram DNA in carboplatin-treated and control wells.

FIG. 6: A graph showing OCC1-SEAP cell response to cisplatin chemotherapy, in vivo as described in Example I, infra.

FIG. 7: A graph showing the effects of carboplatin on tumor burden, as measured by SEAP levels in nude mice, as described in Example I, infra. The arrows indicate times when mice were treated with carboplatin.

FIG. 8: Ovarian tumor cell survival following carboplatin and triptolide treatment, as described in Example II, infra. OCC1-SEAP ovarian cancer cells were incubated for 48 hours in the absence or presence of triptolide (100 ng/ml) and with or without carboplatin (50 mg/ml). Cells remaining in the culture wells following the treatment period were suspended in PBS. The amount of DNA in aliquots of PBS was measured fluorometrically with ethidium bromide and considered representative of amount of cells surviving in culture. The results are expressed as the mean ±SEM, n=4 and are representative of three different experiments.

FIG. 9: DNA fragmentation in OCC1-SEAP cell cultures in response to triptolide and carboplatin treatment, as described in Example II, infra. OCC1-SEAP cell cultures were incubated in the absence or presence of either triptolide (100 ng/ml) or carboplatin (50 mg/ml) as well as combined treatments. DNA was extracted using a PuregeneTM DNA isolation kit and separated by electrophoresis on a 1.2% agarose gel. Low molecular weight DNA fragments were visualized with ethidium bromide stain. Data is representative of three separate experiments.

FIG. 10: Activation of the proteolytic caspase cascade in response to triptolide treatment in OCC1-SEAP cell cultures, as described in Example II, infra. Cell cultures were incubated in the presence of either TPL (100 ng/ml) or carboplatin (50 mg/ml) or a combination of both for 24 h. Aliquots of total cell lysates from cells incubated with treatments were separated by SDS-PAGE and transferred to nylon membranes. Membranes were probed with an antibody to the cleaved (active) (35 and 17 kDa) and full-length (35 kDa) inactive forms of caspase 3 (a-Active caspase 3 and a-caspase 3, respectively). Data are representative of three different experiments.

FIG. 11: Intraperitoneal tumor progression in response to a high dose and short duration of triptolide treatment, as described in Example II, infra. Values presented are SEAP values from post-treatment tumor growth and Day 0 is the first day of treatment initiation. Values are the mean ±SEM with n=3 mice per group.

FIG. 12: Interperitoneal tumor progression in response to a low dose and long duration of triptolide treatment, as described in Example II, infra. Values presented are SEAP values from post-treatment tumor growth and Day 0 is the first day of treatment initiation. Values are the mean ±SEM with n=4 mice per group.

FIG. 13: Interperitoneal tumor progression in response to low dose and long duration of triptolide treatment combined with LY294002 and carboplatin, as described in Example II, infra. Values presented are SEAP values from post-treatment tumor growth and Day 0 with n=6 mice in the control group and n=9 mice in the treated group. An asterisk indicates a significant difference between control and treated groups by Bonferroni post-hock test after 2-way ANOVA. ANOVA showed p=0.064 for treatment, p<0.0001 for day, and p=0.004 for interaction.

FIG. 14: Interperitoneal tumor progression in A) control mice and in B) mice receiving combined triptolide, LY294002 and carboplatin treatment, as described in Example II, infra. Mice were treated with either vehicle control or carboplatin (60 mg/kg) (every other day for 5 days), triptolide (0.15 mg/kg) (every day for 10 days) and LY294002 (40 mg/kg every 2 days for 5 days. Values presented are SEAP values from post-treatment tumor growth, and Day 0 is the first day of treatment initiation. Each line follows the tumor progression of one mouse. Data are from three experiments with n=6 mice in the control group and n=9 mice in the treatment group.

FIG. 15: Interperitoneal tumor progression in representative treated mice with tumor regression in A) relative tumor burden units and in B) SEAP levels, as described in Example II, infra. Mice were treated with carboplatin (60 mg/kg) (every other day for 5 days), triptolide (0.15 mg/kg) (every day for 10 days) and LY294002 (40 mg/kg every 2 days for 5 days). Values presented are SEAP values from post-treatment tumor growth and Day 0 is the first day of treatment initiation. Each line follows the tumor progression of one mouse. Data are from three representative mice.

FIG. 16: Survival curves for control mice versus mice receiving combined triptolide, LY294002 and carboplatin treatment, as described in Example II, infra. The curves reflect when animals were sacrificed or died due to tumor progression. Mice were treated with carboplatin (60 mg/kg) (every other day for 5 days), triptolide (0.15 mg/kg) (every day for 10 days), and LY294002 (40 mg/kg) (every 2 days for 5 days). The curves for control and treated animals are significantly (p=0.03) different as determined by the Mantel-Hanszel logrank test.

FIG. 17: DNA Synthesis in cultures of OCC1-SEAP-12 cancer cells following LY294002 and carboplatin treatment as described in Example III, infra. Data is presented as the mean ±SEM from 4 different experiments.

FIG. 18: Akt phosphorylation in response to PI 3-kinase inhibition in OCC1-SEAP-12 cell cultures, as described in Example III, infra. Top panel: Cell cultures were incubated in the absence (−) or presence (+) of either LY294002 (10 mM) or carboplatin (50 mg/ml) or a combination of both for 24 hr. Bottom panel: Graphic representation of densitometry readings of Phospho-Akt and Akt western blots from 3 separate experiments. Data is presented as the mean ±SEM.

FIG. 19: DNA fragmentation in OCC1-SEAP-12 cell cultures in response to LY294002 and carboplatin treatment, as described in Example III, infra. OCC1-SEAP-12 cell cultures were incubated in the absence (−) or presence (+) of either LY294002 or carboplatin as well as combined treatments for 72 hr. Data are representative of 3 separate experiments.

FIG. 20: Activation of the proteolytic caspase cascade in response to PI 3-kinase inhibition in OCC1-SEAP-12 cell cultures, as described in Example III, infra. Cell cultures were incubated in the absence (−) or presence (+) of either LY294002 (10 μM) or carboplatin (50 μg/ml) or a combination of both for 24 hr. These data are representative of 3 separate experiments.

FIG. 21: Ovarian tumor cell survival following treatment with carboplatin and a PI 3-kinase inhibitor as described in Example III, infra. OCC1-SEAP-12 ovarian cancer cells were incubated for 48 hours in the presence or absence of LY294002 (5 μM and 10 μM) with or without carboplatin (0-50 μg/ml). Data is presented as the mean ±SEM from 4 different experiments.

FIG. 22: Interperitoneal tumor progression in response to carboplatin treatment and PI 3-kinase inhibition, as described in Example III, infra. Mice were treated with either vehicle control, carboplatin (60 mg/kg), LY294002 (50 mg/kg) or a combined carboplatin and LY294002 (Co+LY29) every other day for 6 days. Values presented are SEAP values from post-treatment tumor growth, and Day 0 is the last day of treatment injection. Values are the mean ±SEM, n=9 or 10 mice per treatment group.

FIG. 23: Effects of LY294002 and carboplatin on tumor growth in mice inoculated with OCC1-SEAP-12 ovarian cancer cells, as described in Example III, infra. Mice were treated with either vehicle control, carboplatin (60 mg/kg), LY294002 (50 mg/kg) or combined carboplatin and LY294002 (Co+LY29) every other day for 6 days. Values represent relative final tumor burden (as measured by plasma levels of SEAP) at last common bleed. Values are the mean ±SEM, n=9 or 10 mice per group.

FIG. 24: Appearance of mice after treatment with LY294002 and carboplatin alone and in combination as described in Example III, infra. Four representative nude mice inoculated with OCC1-SEAP-12 cells and treated with either vehicle control (PBS+DMSO) only (A); with carboplatin (60 mg/kg) alone (B); with LY294002 (50 mg/kg) alone (C); or with carboplatin+LY294002 (D).

### DETAILED DESCRIPTION OF THE INVENTION

All scientific and technical terms used in this application have meanings commonly used in the art unless otherwise specified. As used in this application, the following words or phrases have the meanings specified.

Definitions

The terms “cellular process” or “cellular processes” as used herein, include, but are not limited to, cellular proliferation, differentiation, apoptosis, adhesion, motility, neovascularization, viability, senescence, metabolism, DNA replication, gene transcription, and RNA translation. Cellular processes also include abnormal cellular processes, including transformation, blocking of differentiation, and metastasis, which are caused by abnormal or uncontrolled cellular processes, signal transduction pathways, or factors that mediate cellular processes or signal transduction pathways.

The term “signal transduction” as used herein, means a transmission of cellular signals from the exterior to the interior of a cell via a pathway of interactive proteins known as “signaling proteins”. Signaling proteins include exterior and interior proteins, such as cell surface proteins, transmembrane proteins, intracellular proteins, and nuclear proteins. Many of the signaling proteins transmit cellular signals throughout the cell using a reversible phosphorylation mechanism of amino acid residues on the signaling proteins. For example, reversible phosphorylation of tyrosine, serine or threonine residues plays a key role in transmitting signals throughout the cell.

The term “cellular factors” as used herein, includes transmembrane proteins, intracellular proteins and nucleic acid molecules, and nuclear proteins and nucleic acid molecules. Cellular factors include kinases and phosphatases. The cellular factors that mediate the PI3K pathway include, but are not limited to, PI3K, p85, p110, Akt, or BAD.

The terms “modulate” and “modulation” as used herein, mean upregulating or downregulating a cellular process, or a signal transduction pathway, or a PI3K signal transduction pathway. Modulation also includes increasing or decreasing the activity of the cellular factors that mediate a cellular process, or that mediate a signal transduction pathway, or that mediate the PI3K signal transduction pathway.

The term “antagonist” as used herein means an agent that mimics the effects of a cellular, endogenous regulatory compound. An antagonistic agent has intrinsic regulatory activity.

The term “agonist” means an agent that inhibits the action of an antagonist, such as, for example by competing with an antagonist for a binding site. An agonist does not have any intrinsic regulatory activity.

Compositions

The present invention provides compositions and methods for treating a subject, having a disease condition or cancer, caused by a dysfunctional PI3K signal transduction pathway.

The compositions of the invention inhibit growth of or kill cancer cells, by modulating the activity of cellular factors, that mediate the PI3K signal transduction pathway.

In one embodiment, the compositions of the invention comprise an agent that inhibits the PI3K signal transduction pathway, in combination with triptolide.

In another embodiment, the compositions of the invention comprise an agent that inhibits the PI3K signal transduction pathway, and a chemotherapy agent such as carboplatin, in combination with triptolide.

The signal transduction pathways, include, but are not limited to, the PI3K pathway (Carpenter, Duckworth et al. 1990; Pignataro and Ascoli 1990; Varticovski, Harrison-Findik et al. 1994; Hu, Klippel et al. 1995; Yao and Cooper 1995; Minshall, Arkins et al. 1996; Yao and Cooper 1996; Grasso, Wen et al. 1997).

The present invention provides compositions comprising an inhibitor of the PI3K signal transduction pathway, and/or an antagonist of lysophosphatidic acid (LPA).

The LPA antagonist can be LPA 10:0, LPA 14:0, or LXR LPA.

The present invention provides compositions comprising an inhibitor of the PI3K signal transduction pathway, and triptolide.

In one embodiment, the inhibitor of the PI3K signal transduction pathway is PD098059 or U0126. In another embodiment, the LPA antagonist is LPA 10:0, LPA14:0, or LXR LPA.

In one embodiment, the present invention provides compositions comprising PD09059 and/or U0126, and an LPA antagonist, such as LPA 10:0, LPA 14:0, or LXR LPA.

In another embodiment, the present invention provides compositions comprising LY294002 and/or Wortmannin, and LPA antagonist such as LPA 10:0, LPA 14:0, or LXR LPA.

In another embodiment, the present invention provides compositions comprising PD09059 and/or U0126, and LY294002 and/or Wortmannin, and an LPA antagonist such as LPA 10:0, LPA 14:0, or LXR LPA.

In another embodiment, the present invention provides compositions comprising PD09059 and/or U0126, and triptolide.

In another embodiment, the present invention provides compositions comprising LY294002 and/or Wortmannin, and triptolide.

In another embodiment, the present invention provides compositions comprising PD09059 and/or U0126, and LY294002 and/or Wortmannin, and triptolide.

Pharmaceutical Compositions

The present invention provides pharmaceutical compositions, comprising the compositions of the invention and a pharmaceutical carrier. The pharmaceutical compositions of the invention are formulated by well known conventional methods. The present invention also provides methods for formulating pharmaceutical compositions comprising the compositions of the invention.

The pharmaceutical carrier includes phosphate buffered saline solution, water, oil/water emulsion, mineral oil, wetting agent, sterile solution, excipients, starch, milk, sugar, lactose, dextrose, sucrose, sorbitol, mannitol, gum acacia, alginates, tragacanth, gelatin, clay, gelatin, stearic acid, magnesium stearate, calcium stearate, calcium phosphate, calcium silicate, microcrystalline cellulose, polyvinylpyrrolidone, cellulose, water syrup, methyl cellulose, methyl and propylhydroxybenzoates, propylene glycol, liquid paraffin, white soft paraffin, kaolin, microcrystalline cellulose, calcium silicate, silica, cetostearyl alcohol, cocoa butter, oil of theobroma, arachis oil, syrup B.P., methyl cellulose, polyoxyethylene sorbitan monolaurate, ethyl lactate, propylhydroxybenzoate, sorbitan trioleate, sorbitan sesquioleate, oleyl alcohol, talc, vegetable fat, vegetable oil, gum, or glycol. The compositions are formulated using lubricating agents, wetting agents, emulsifying agents, preservatives, sweetening, flavoring, or coloring agents.

The pharmaceutical compositions of the invention are formulated as a pill, tablet, coated tablet, capsule, liposome, polymeric microsphere, patch, powders, lozenges, sachets, cachets, elixirs, suspensions, emulsions, solutions, syrups, aerosol (as a solid or in a liquid medium), soft or hard gelatin capsules, suppositories, sterile injectable solutions and sterile packaged powders for either oral or topical application.

In one embodiment, the compositions are formulated as a dipalmitoyl phosphatidyl choline liposome (DPPL) (Simon, Hicks et al. 1999). In another embodiment, the compositions are formulated in DMSO (Hu, Zaloudek et al. 2000).

The pharmaceutical compositions of the present invention may be manufactured by well-known methods, including conventional mixing, dissolving, granulating, dragee-making, levigating, emulsifying, encapsulating, entrapping or lyophilizing processes.

Proper formulation of the pharmaceutical compositions is dependent upon the route of administration chosen. For injection, the pharmaceutical compositions of the invention may be formulated in aqueous solutions, in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the pharmaceutical compositions can be formulated by combining the active compounds with pharmaceutically acceptable carriers well known in the art. Such carriers enable the compounds of the invention to be formulated as tablets, pills, dragees, capsules, liquids, gels, syrups, slurries, suspensions and the like. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). Disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration are preferably in dosages suitable for such administration.

For buccal administration, the pharmaceutical compositions may take the form of tablets or lozenges formulated in conventional manner.

For administration by inhalation, the pharmaceutical compositions are delivered in the form of an aerosol spray presentation from pressurized packs or a nebulizer, with the use of a suitable propellant, including dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

The compositions of the invention are formulated as injectable compositions according to conventional methods using suitable dispensing, suspending, or wetting agents. The compositions are formulated as sterile injectable aqueous or oleaginous suspensions. The compositions are formulated as a suspension in a nontoxic parenterally acceptable diluent or solvent, for example, as a solution in 1,3-butanediol. Techniques for formulation and administration of the compositions of the invention may be found in “Remington's Pharmaceutical Sciences,” Mack Publishing Co., Easton, Pa.

Alternatively, the active ingredient may be in powder form for constitution with a suitable vehicle, such as sterile pyrogen-free water, before use.

The pharmaceutical compositions may also be formulated in rectal compositions such as suppositories or retention enemas, e.g., containing conventional suppository bases such as cocoa butter or other glycerides.

In addition to the formulations described previously, the compositions may also be formulated as a depot preparation. Such long acting formulations may be administered by implantation (for example subcutaneously or intramuscularly) or by intramuscular injection. The compositions may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. The identity of the co-solvent components may be varied: for example, other low-toxicity nonpolar surfactants may be used instead of polysorbate 80. The fraction size of polyethylene glycol may be varied. Other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semi permeable matrices of solid hydrophobic polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

The compounds of the present invention may be administered by controlled release means and/or delivery devices including Alzet (a registered trademark of Alza, Corporation) osmotic pumps. Suitable delivery devices are described in U.S. Pat. Nos. 3,845,770; 3,916,899; 3,536,809; 3,598,123; 3,944,064; and 4,008,719, the disclosures of which are incorporated in their entirety by reference herein. Other routes of administration include targeted drug delivery systems, for example, in a liposome coated with a tumor-specific antibody. The liposomes will selectively target the tumor.

Many of the phosphatase modulating compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, and the like. Salts tend to be more soluble in aqueous or other protonic solvents that are the corresponding free base forms.

Methods

The present invention provides methods for modulating cellular processes, or modulating signal transduction pathways, or modulating the activity of cellular factors that mediate cellular processes, or modulating the PI3K signal transduction pathway. The methods of the invention may be practiced on cultured cells, or by administering to a subject. The present invention also provides methods for treating a subject having a condition, such as cancer, caused by a dysfunctional signal transduction pathway. Additionally, the present invention provides methods for inhibiting growth of, and/or killing, cancer cells in a subject.

The methods of the invention comprise administering to the cells, or to a subject, a therapeutically effective amount of a composition of the invention. The therapeutically effective amount, is an amount of composition, for a time that is sufficient to inhibit cell growth of and/or kill the cells.

In one embodiment, the cells or the cells in the subject, include various types of cancer cells, including solid tumors neoplasms, sarcomas, and carcinomas. The cancer cells include cancer cells from the ovary, lung, mammary, breast, prostate, testicular, pancreatic, bladder, colon, head and neck, esophageal, hepatoma, lymphoma, epidermoid carcinoma, glioblastoma, melanoma, and Kaposi's sarcoma. The diseased cells are from a subject having diseases including, but not limited to, diabetes, diabetic retinopathy, rheumatoid arthritis, hemangioma, or leukemia, including promyelocytic leukemia.

The compositions of the method can be introduced to cells from a human, ape, monkey, equine, porcine, bovine, murine, canine, feline, or avian subject.

The present invention also provides methods for enhancing the sensitivity of a cancer cell to the growth inhibiting, or killing effects of chemotherapeutic agents, hormone therapy or radiation therapy. The methods comprise administering to a subject a therapeutically effective amount of a composition of the invention, in combination with a therapeutically effective amount of a chemotherapeutic agent and/or in combination with a hormone regimen, and/or in combination with a radiotherapy method.

Particular embodiments include administering to a subject a therapeutically effective amount of a chemotherapeutic agent, and/or a hormone regimen and/or a radiotherapy method, in combination with a therapeutically effective amount of a composition of the invention, including but not limited to, an inhibitor of the PI3K and an antagonist of lysophosphatidic acid (LPA); an inhibitor of the PI3K signal transduction pathway(s) and triptolide.

The chemotherapeutic agent includes any chemical compound or drug that inhibits growth or kills tumor cells, including, but not limited to, platinum-containing drugs, Taxol or derivatives, or cyclophosphamide. The platinum-containing drugs include cisplatin, carboplatin, oxaliplatin, and the like. Derivatives of Taxol include paclitaxel and the like.

The radiation therapy includes exposing the subject to ionizing radiation. In one embodiment, the radiation therapy includes gamma-radiation. Such radiation therapy is routinely practiced by those skilled in the art. Protocols for administering drugs or agents in combination with radiation therapy have been established (Wobst, Audisio et al. 1998). The dose of ionizing radiation will vary depending on a variety of factors including intensity, source of radiation, site to be treated, and the like.

In the methods of the invention, the agents that comprise the compositions of the invention are administered to the subject together as an admixture, or administered together separately but simultaneously, or substantially simultaneously, or the two agents are administered sequentially. The compositions of the present invention can be administered to a subject prior to, simultaneously, substantially simultaneously, or sequentially with other therapeutic regimens.

Dosage

A therapeutically effective amount of the compositions of the invention is administered to a subject is an amount and for a time sufficient to inhibit cell growth or kill a cancer cell, or to enhance the cell-killing effect of the chemotherapy and/or radiation therapy and/or hormone therapy.

A therapeutically effective amount may vary depending on the gender, age, weight and condition of the subject, and is determined on a case-by-case basis. An effective amount may vary according to the size and type of cancer present. An effective amount may vary depending on the type of therapeutic regimen administered. For example, determination of effective amounts of the compositions of the invention is to be administered is well within the capability of those skilled in the art.

The therapeutically effective amount can be estimated initially from cell culture assays. Alternatively, the therapeutically effective amount can be determined in an animal model to achieve a circulating concentration range that includes the IC50, as determined in cell culture (i.e., the concentration of the composition of the invention which achieves a half-maximal cell growth inhibition or cell killing). Such information can be used to more accurately determine the therapeutically effective doses in humans.

Toxicity and therapeutic efficacy of the inventive compositions are determined by standard pharmaceutical procedures, in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio between LD50 and ED50. The dosage may vary depending upon the dosage form employed and the route of administration utilized. The exact formulation, route of administration and dosage can be chosen by a physician in view of the patient's condition. (Fingl and Woodbury 1975).

Dosage amount and interval may be adjusted individually to provide plasma levels of the active moiety which are sufficient to maintain the phosphatase modulating effects, or minimal effective concentration (MEC). The MEC will vary for each compound but can be estimated from in vitro data; e.g., the concentration necessary to achieve a 50-90% inhibition of the phosphatase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90%.

Desirable blood levels may be maintained by a continuous infusion of the compound as ascertained by plasma levels measured by HPLC. It should be noted that the attending physician would know how to and when to terminate, interrupt or adjust therapy to lower dosage due to toxicity, or bone marrow, liver or kidney dysfunctions. Conversely, the attending physician would also know to adjust treatment to higher levels if the clinical response is not adequate, precluding toxicity.

Usual patient dosages for systemic administration range from 1 to 2000 mg/day of the compositions of the invention, commonly from 1 to 250 mg/day, and typically from 10 to 150 mg/day. Stated in terms of patient body weight, usual dosages range from 0.02 to 25 mg/kg/day, commonly from 0.02 to 3 mg/kg/day, typically from 0.2 to 1.5 mg/kg/day. Stated in terms of patient body surface areas, usual dosages range from 0.5 to 1200 mg/meter²/day, commonly from 0.5 to 150 mg/meter²/day, typically from 5 to 100 mg/meter²/day. Usual average plasma levels should be maintained within 50 to 5000 micro g/ml, commonly 50 to 1000 micro g/ml, and typically 100 to 500 micro g/ml. It is further recommended that infants, children, and patients over 65 years, and those with impaired renal, or hepatic function, initially receive low doses, and that they be titrated based on individual clinical response(s) and blood level(s). It may be necessary to use dosages outside these ranges in some cases as will be apparent to those of ordinary skill in the art.

Generally, a suitable dose is one that results in a concentration of the composition of the invention at the site of the tumor in the range of 0.5 nM to 200 micro M, and more usually from 20 nM to 80 nM. It is expected that serum concentrations from 40 nM to 150 nM should be sufficient in most cases.

In cases of local administration or selective uptake, the effective local concentration of the composition may not be related to plasma concentration.

The magnitude of a prophylactic or therapeutic dose of the compound in the acute or chronic management of disease will vary with the severity of the condition to be treated and the route of administration. Again, it should be noted that the clinician or physician will know when to interrupt and/or adjust the treatment dose due to toxicity or bone marrow, liver or kidney dysfunctions. The dose, and perhaps the dosage frequency, will also vary according to the age, body weight, and response of the individual patient. In general, as discussed above, the total daily dose ranges for the compounds for the majority of the disorders described herein, is from about 0.02 to about 25 mg/kg patient. Preferably, a daily dose range should be between about 0.02 to about 3 mg/kg, while most preferably a daily dose range should be between about 0.2 to about 1.5 mg/kg per day.

Animal Model for Ovarian Cancer

The present invention provides methods used in an animal model for ovarian cancer comprising an animal host harboring a tumor cell line expressing a reporter gene, where expression of the reporter gene product correlates with tumor burden within the animal.

An animal model harboring a tumor cell line expressing a reporter gene has previously been reported. This animal model is a SCID mouse transplanted subcutaneously or intraperitoneally with ovarian carcinoma line A2780, where the ovarian carcinoma is transiently or stably transfected with a heat-stable, human SEAP gene (Bao, Selvakumaran et al. 2000). A nude mouse tumor model was developed that has been shown to be effective in evaluating the response of ovarian tumor xenografts in nude mice (Nilsson, Westfall et al. 2002). This in vivo tumor model involves a human ovarian cancer cell line, OCC1, which has been stably transfected with a secreted alkaline phosphatase (SEAP) gene. The SEAP reporter gene is constitutively expressed and has been shown to be secreted at levels proportional to the number of tumor cells present (Nilsson, Westfall et al. 2002).

In one embodiment, the animal host is ape, monkey, equine, porcine, bovine, murine, canine, or feline. In a preferred embodiment, the animal is a mouse or rat. The animal can be immunocompromised, including animals that are severe combined immunodeficient (e.g., SCID), or athymic nude.

The tumor cell line can be from cancer cells from the ovary, lung, mammary, breast, prostate, testicular, pancreatic, bladder, colon, head and neck, esophogeal, hepatoma, lymphoma, epidermoid carcinoma, glioblastoma, melanoma, Kaposi's sarcoma.

The tumor cell line harbored by the animal can be transplanted from any type of animal including human, ape, monkey, equine, porcine, bovine, murine, mouse, rat, canine, or feline.

The tumor cell line can be an ovarian tumor cell line, including chemo-sensitive or chemo-resistant lines. The chemo-sensitive lines include A2780-s, OV2009, and OVCAR-3. The chemo-resistant lines include A2780cp. In a preferred embodiment, the tumor cell line is a human, ovarian tumor cell line A2789 or OCC1 (Wong, Wong et al. 1990) or SKOV3.

The transplanted tumor cell line is a transgenic cell line which carries a reporter gene. The reporter gene product becomes distributed throughout the animal body at levels proportional to the number of tumor cells in the animal. The reporter gene can be secreted placental alkaline phosphatase protein (e.g., SEAP) (Berger, Hauber et al. 1988). In a preferred embodiment, the reporter gene is heat-stable, human placental SEAP (Berger, Hauber et al. 1988; Nilsson, Westfall et al. 2002). The reporter gene can be expressed in an inducible or constitutive manner. In a preferred embodiment, the expression of the reporter gene is controlled by a constitutive promoter or enhancer sequence, such as an SV40 enhancer sequence. The transgenic cell line can be transiently or stably transfected with the reporter gene using known transfection methods.

The transgenic tumor cell line can be transplanted into the animal via injection subcutaneously or intraperitoneally. For ovarian tumor cell lines, intraperitoneal methods are preferred.

The animal model is useful for monitoring the effectiveness of novel or known therapeutic agents, and regimens for inhibiting growth of tumor cells, or killing tumor cells in an animal.

In a preferred embodiment, the animal model is an athymic nude mouse transplanted intraperitoneally with human ovarian carcinoma line OCC1, where the ovarian carcinoma is stably transfected to express a heat-stable, human SEAP gene (Nilsson, Westfall et al. 2002). SEAP is distributed through the body at levels proportional to the number of tumor cells in the animal. The SEAP protein is detectable in small blood samples collected into capillary tubes. Animals are repeatedly sampled over the trial period to monitor the course of tumor progression.

Developing new anticancer therapeutic regimens requires the measurement of tumor cell growth (inhibition), in response to treatment. This is often accomplished by injecting athymic nude mice, or other susceptible animals, with cells from cancer tissue or cell lines. After treating the animals with the chemotherapeutic agent, the tumor weight or volume is measured at the end of the experiment. This method is complicated by inaccuracies in measuring tumor weight and volume. Additionally, the animal is often killed to measure tumor burden.

For tumors that primarily grow intraperitoneally in their host, such as ovarian carcinomas, the most appropriate experimental animal model is to inject and grow the tumor cells intraperitoneally in the mouse. Measuring intraperitoneal tumor growth and response to treatment in a living animal is difficult. Changes in body weight are difficult to measure because of the diluting effect of the weight of the animal itself and are complicated by weight loss due to tumor cachexia or anticancer drug therapy. Abdominal tumors may be dissected out of a host animal and weighed, but it is often difficult to find and isolate all the tumor mass from the host tissue. This procedure is also complicated by the fact that some tumors, including ovarian carcinomas, recruit host cells into the tumor itself (Parrott, Nilsson et al. 2001). In addition, the animal must be killed to dissect and measure an intraperitoneal tumor. Therefore tumor size is only measured at the end of the trial, and may not be evaluated over the course of therapy.

The growth of subcutaneous tumors may be followed over time in a host animal by measuring the diameter of the subcutaneous mass. However, this method is sometimes inaccurate if the tumor is invasive and grows into the underlying tissue rather than spreading under the skin. Additional inaccuracy is introduced if the tumor forms a necrotic center. As mentioned above, tumors such as ovarian carcinomas are more appropriately grown intraperitoneally than subcutaneously to examine tumor progression.

Methods for Inhibiting Growth of or Killing Tumor Cells in a Subject

The present invention provides methods for inhibiting the growth of tumor cells and/or killing tumor cells in a subject. The present invention also provides methods for monitoring tumor growth (e.g., tumor burden) and/or the killing of tumor cells in a subject.

The methods of the invention include using an animal model to test the efficacy of therapeutic agents, where the animal harbors a tumor cell line expressing a reporter gene, and expression of the reporter gene product correlates with tumor burden within the animal.

The methods comprise administering to a subject, a therapeutically effective composition of the invention, in combination with a therapeutic regimen that inhibits growth of tumor cells and/or kills tumor cells, to inhibit tumor growth and/or kill tumor cells.

The therapeutic regimen includes chemotherapy, radiation therapy, a hormone regimen, or any combination of these therapies. In one embodiment, the animal is treated with platinum-containing, chemotherapy compounds including cisplatin, carboplatin and/or oxaliplatin, and/or with cyclophosphamide, and/or with Taxol, or paclitaxel. In another embodiment, the animal is treated with a chemotherapy regimen comprising a composition of the present invention, e.g., an agent that is an inhibitor of the PI3K signal transduction pathway and triptolide.

The present invention provides methods for monitoring tumor growth and/or killing tumor cells, comprising measuring, in a sample from an animal model, the amount of RNA transcript or protein product encoded by the reporter gene, in order to monitor tumor growth inhibition, and/or tumor cell killing. A method of the invention comprises measuring the amount of SEAP RNA transcript or SEAP protein in a sample from the animal. The level of the RNA transcript or protein product encoded by the reporter gene is measured in a sample of blood from the animal. In another embodiment, the monitoring methods comprise measuring the size, volume and/or weight of the tumor, using techniques well known in the art.

The following examples are presented to illustrate the present invention and to assist one of ordinary skill in making and using the same. The methodology and results may vary depending on the intended goal of treatment and the procedures employed. The examples are not intended in any way to otherwise limit the scope of the invention.

### EXAMPLE I

The following provides a description of methods used to create the animal model used to monitor ovarian tumor growth. The animal model was then used to monitor ovarian tumor growth in response to compositions of the invention.

Materials and Methods

Transfection of SEAP Gene into OCC1 Cells

The expression vector, pCMV-SEAP, includes the SEAP gene and SV40 enhancer derived from p-SEAP2-enhancer plasmid (Clontech, Palo Alto, Calif.) cloned into pcDNA3. The pCMV-SEAP plasmid was transfected into the OCC1 (Wong, Wong et al. 1990) ovarian carcinoma cell line using transfection mediated by Fugene 6 (Boehringer Mannheim). To each well of cells in a 24-well plate were added 200 ng plasmid DNA and 1 micro liter Fugene 6 reagent. Cells with stably integrated SEAP were selected for neomycin resistance by treating with 600 microgram/ml G418 (Cal-Biochem, La Jolla, Calif.). Clonal isolates were grown and culture medium tested for SEAP production. Of 18 clonal isolates of OCC1, one produced very high levels of SEAP (OCC1-SEAP-12). OCC1-SEAP-12 was used in subsequent in vivo and in vitro experiments.

OCC1-SEAP-12 cells were grown in Ham's F-12 medium (Gibco) plus 10% calf serum then collected into Hank's balanced salt solution and counted for injection into nude mice. For in vitro assays OCC1-SEAP-12 cells were plated at sequential twofold dilutions and allowed to adhere to culture wells and incubated for 24 hours. Alternatively, OCC1-SEAP-12 cells were grown to 80% confluence in Ham's F-12 medium plus 10% calf serum. Cells were starved for 48 hours in DMEM plus 0.1% BSA and 0.1% calf serum. Experimental treatments were applied for 2 days in DMEM plus 0.1% BSA and 0.1% calf serum. At the end of the treatment period a sample of culture medium was taken for SEAP level determination. DNA assays were performed by discarding the remaining culture medium from over the cells, adding buffer solution, sonicating the cells in each well, and measuring the fluorescence of cell solution into which SYBR Green I fluorescent dye (Molecular Probes, Eugene, Oreg.) had been incorporated as previously described (Parrott and Skinner 1999).

Animal Use and in vitro Treatment Protocols

Athymic nude mice (Nu/Nu; Charles River Laboratories, Wilmington, Mass.) weighing about 25 g were used for in vivo studies.

Four nude mice were injected subcutaneously into the dorsal flank region with 1×10⁷ OCC1-SEAP-12 cells. Measurements of tumor length, width and height were taken three times per week. One-half the average diameter was used as the radius to calculate tumor volume using the equation: volume=(4/3)(πr³). Blood samples were taken for the SEAP assay.

Three mice were injected intraperitoneally with 1×10⁷ OCC1-SEAP-12 cells. The experimental treatments began after 1 week. Animals received intraperitoneal injections of 3 mg/kg cisplatin divided over 2 weeks (one injection per week), 10 mg/kg cisplatin divided over 2 weeks or a vehicle control. Alternatively, four mice with well-developed tumors (2-4 weeks after injection) were treated with a high dose of carboplatin (60 mg/kg) three times over a period of 4 days. After death all visible intraperitoneal tumors were dissected out and weighed.

Blood samples were collected from saphenous vein lancings three times per week into heparinized capillary tubes. The capillary tubes were centrifuged and the plasma samples were frozen at −20° C. until the time of SEAP assay. Blood samples were taken for SEAP assay from the mice within 24 hours of death 2-3 weeks after intraperitoneal injection of OCC1-SEAP cells.

Blood plasma and cell culture medium samples were assayed for SEAP activity using the Great EscAPe SEAP fluorescence detection kit (Clontech Laboratories, Palo Alto, Calif.). SEAP is secreted at a constant rate by transfected tumor cells and distributed throughout the body or into the culture medium. SEAP is heat stable, so any endogenous alkaline phosphatase activity in blood plasma is destroyed by heat treatment of the samples (65° C. for 30 minutes) during the assay. The SEAP enzyme in each sample acts on the substrate 4-methylumbelliferyl phosphate during a 1 hour incubation at room temperature to produce a fluorescent product with excitation and emission peaks at 360 and 449 nm, respectively. A fluorimeter is then used to measure SEAP activity. The intra-assay and inter-assay coefficients of variation were 2.5% and 18.8%, respectively. Blood plasma samples were diluted 1:100 prior to the assay to bring values within the linear range of the standard curve.

To determine the degree of correlations between tumor volume and SEAP levels, a correlation coefficient was calculated from measurements of subcutaneous tumor volume and plasma SEAP levels of samples collected at the same time. A correlation coefficient was calculated for intraperitoneal tumors between plasma SEAP levels of blood samples collected within 24 hours of death and intraperitoneal tumor weight.

Results

The results show a correlation of in vitro response to drug therapy with response to treatment in the live animal, ovarian cancer model. Treatment with platinum-containing drugs does not alter the rate of SEAP secretion per cell. Therefore, the nude mouse model, using SEAP as a marker, is an accurate indicator for monitoring ovarian tumor growth and progression.

Plasma SEAP Concentrations Correlate with Tumor Size

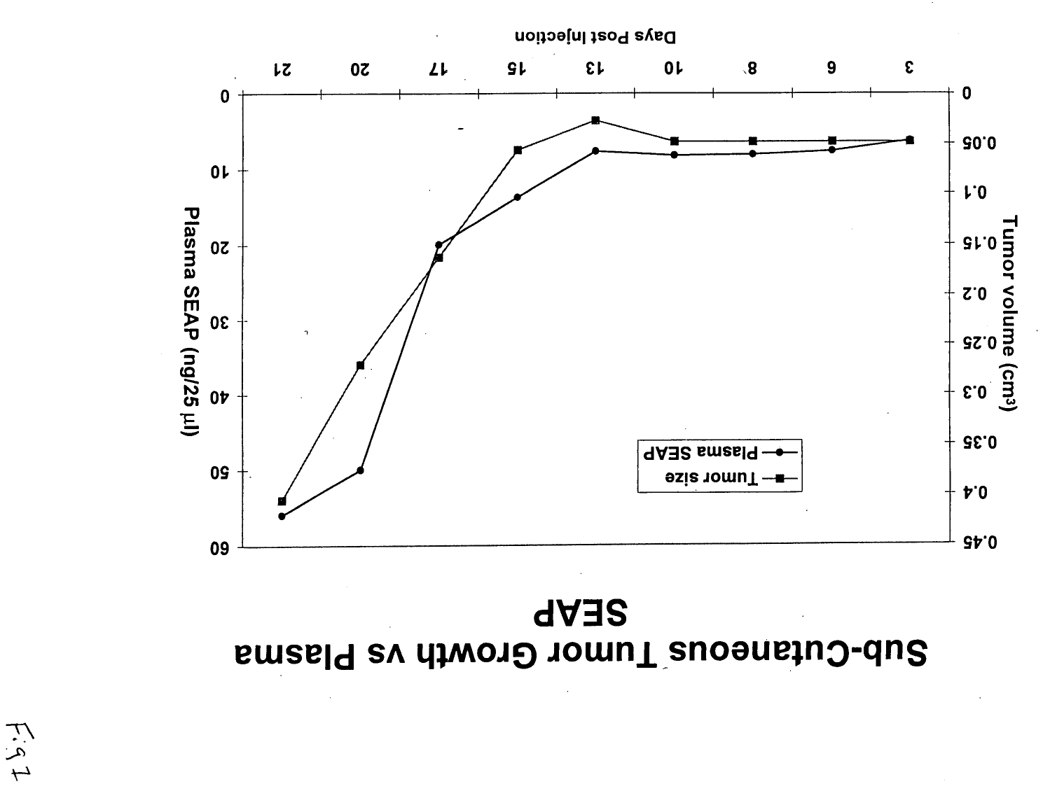

OCC1-SEAP cells were injected subcutaneously into nude mice, and the average diameter of the growing tumor mass was measured three times per week to calculate tumor volume as described above. Blood samples were also taken and plasma SEAP levels were determined (nanograms SEAP per 25 micro liters). While tumor volumes and tumor growth rates varied considerably between mice, SEAP levels were found to correspond closely to calculated tumor volume within individual mice. Results from a representative mouse out of four are shown in FIG. 1. The correlation coefficient was R²=0.95. Representative results from four different mice and two experiments are presented

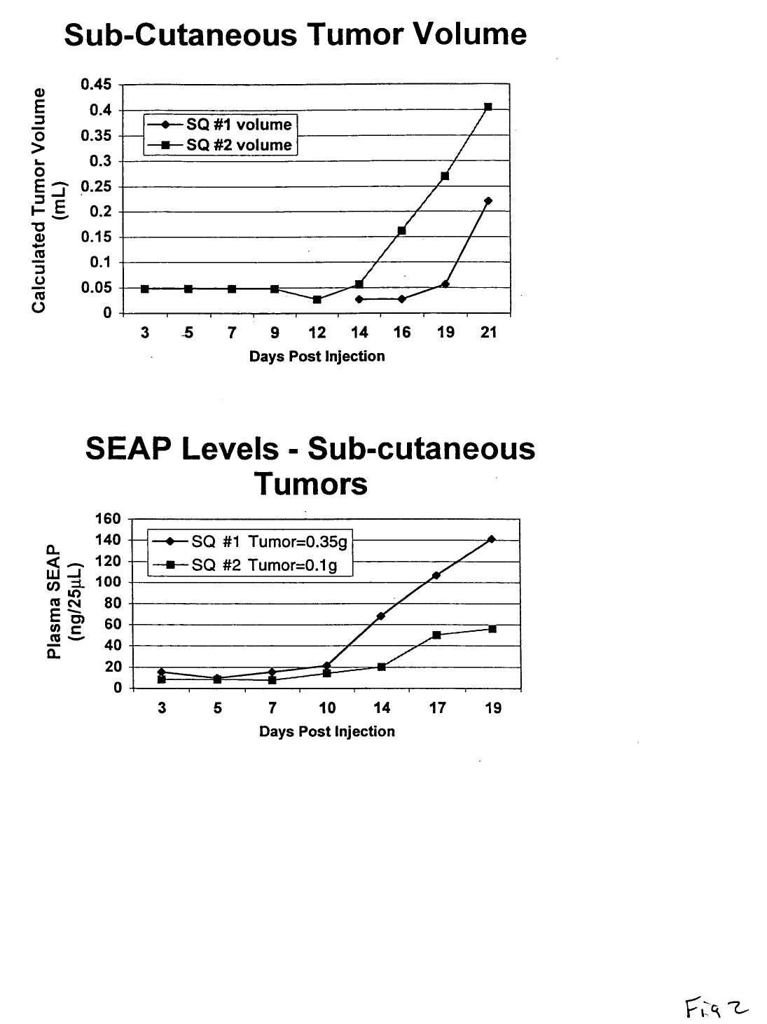

Tumor invasiveness could result in inaccurate subcutaneous tumor volume measurement (FIG. 2). Two mice were injected subcutaneously with OCC1-SEAP cells. The mouse with the smaller tumor volume measurement had higher plasma SEAP levels than the mouse with the apparently larger tumor volume measurement. However, at the termination of the experiment dissection showed that the smaller volume tumor had invaded into the underlying body wall, and actually weighed much more than the tumor in the other mouse. The tumor in SQ#1 had invaded the underlying body wall and was larger (0.35 g) than the tumor in SQ#2 (0.1 g). This indicates that plasma SEAP levels were a more accurate indicator of tumor mass than were tumor diameter measurements.

Correlation Between Tumor Mass and SEAP Levels

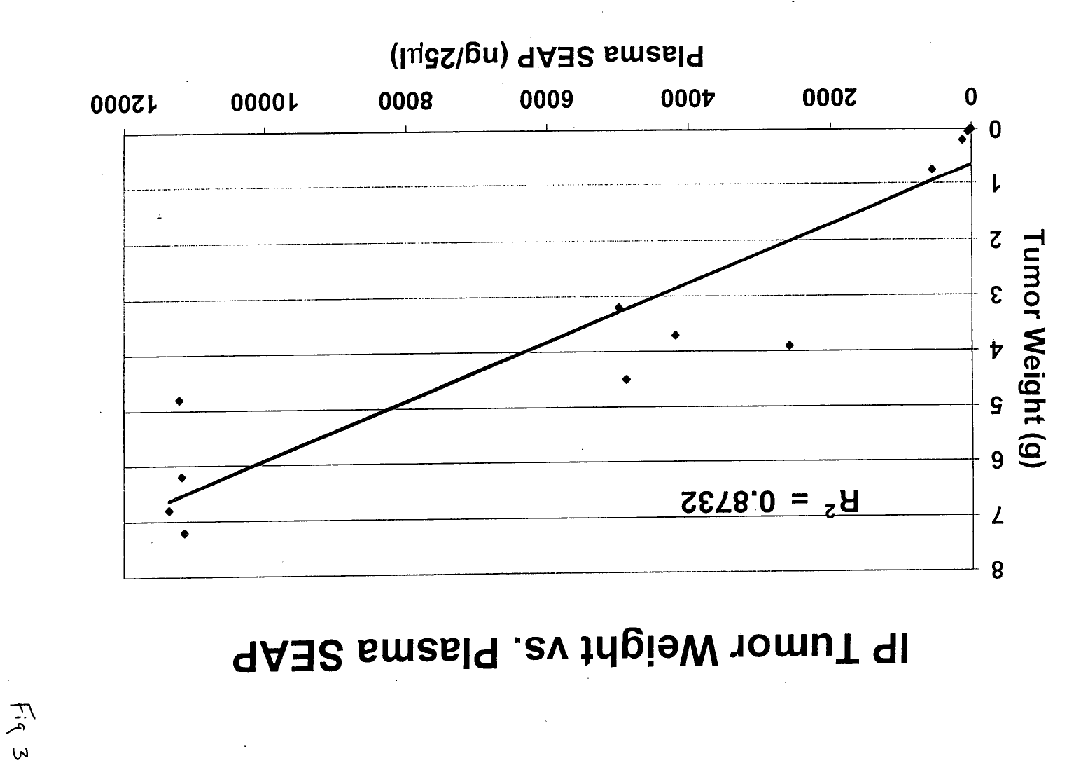

The correlation between intraperitoneal tumor mass and plasma SEAP levels was investigated by injecting OCC1-SEAP cells intraperitoneally into 13 nude mice and letting the tumors develop for approximately 3 weeks. At the end of this time the animals were killed and all visible tumor was dissected from the abdominal cavity and weighed. Blood samples from the time of death were used to determine end-point plasma SEAP levels. The SEAP levels were correlated with tumor weight (R²=0.87; FIG. 3).

OCC1-SEAP Cells Respond to Platinum-containing Drug Treatment in vitro

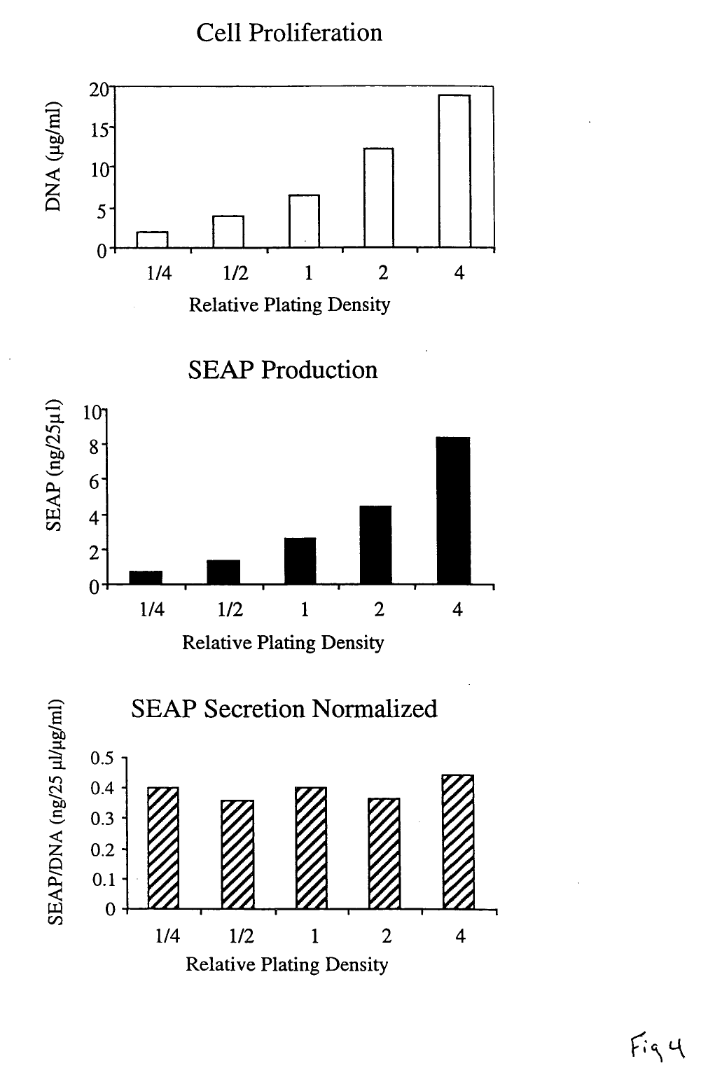

In vitro studies were performed to verify that SEAP levels correspond to cell density after culturing OCC1-SEAP cells for 24 hours SEAP levels in the medium corresponded directly to the amount of DNA present in a culture well (FIG. 4). The rate of SEAP secretion per cell was the same at all cell densities (FIG. 4). This indicates that SEAP levels were an accurate indicator of tumor cell number at different cell densities. OCC1-SEAP cells were cultured in the presence or absence of 60 1 ml carboplatin (Sigma).

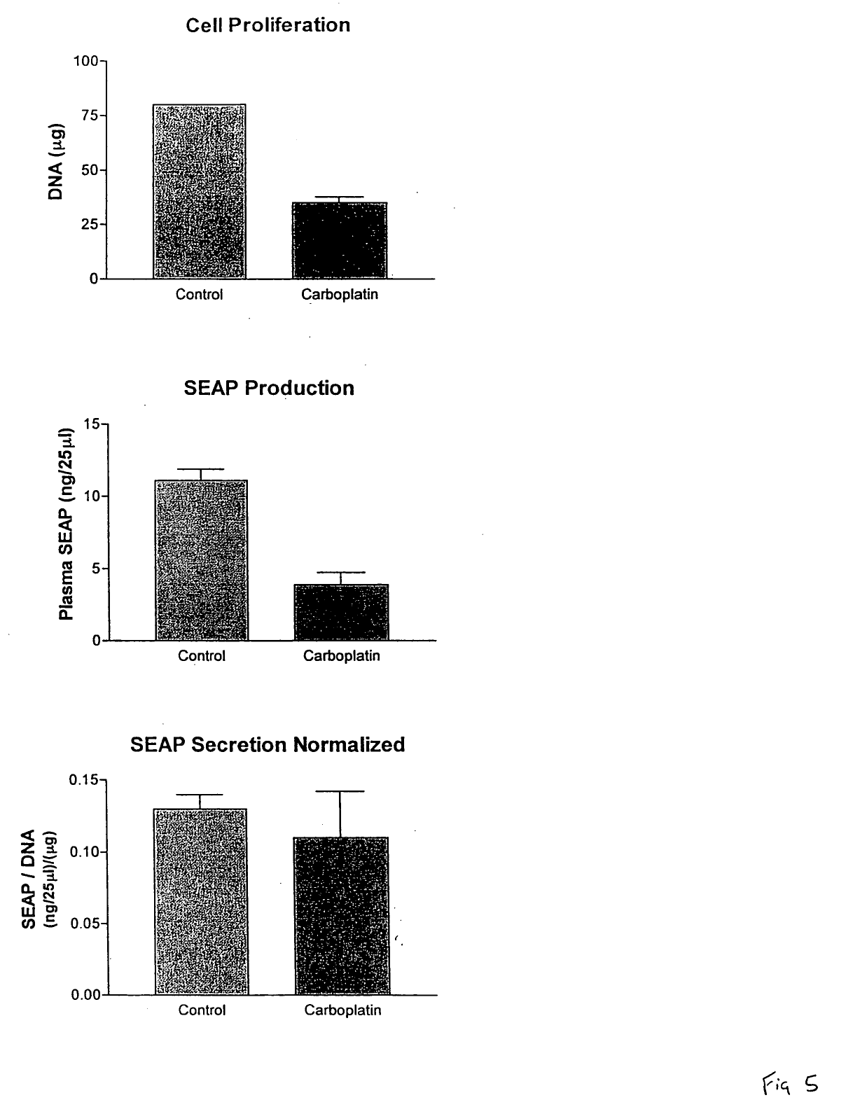

Carboplatin treatment decreased tumor growth in vitro, as measured both by the amount of DNA per well and by the amount of SEAP in the culture medium (FIG. 5). OCC1-SEAP cells were grown to 50% confluence in Ham's F-12 plus 10% calf serum and then starved for 2 days in DMEM plus 0.1% BSA and 0.1% calf serum. Cells were then treated with 60 micro g/ml carboplatin for 48 hours. The bars indicate the means ±SEM from three different experiments. In OCC1-SEAP cells the DNA levels decreased from 58 micro g/ml for control cells to 35 micro g/ml for carboplatin-treated cells. SEAP levels also decreased from 11.1 ng per 25 micro liter in control cells to 3.9 ng per 25 micro liter in carboplatin-treated cells. SEAP production was normalized by the amount of DNA per well (FIG. 5). This ratio reflects the amount of SEAP produced per cell. This normalized SEAP production did not change in the presence or absence of carboplatin treatment. The anticancer drug treatment did not change constitutive SEAP production. Therefore, SEAP could provide a marker for tumor burden independent of carboplatin chemotherapeutic treatment.

OCC1-SEAP Cells Respond to Platinum-containing Drug Treatment in vivo in the Nude Mouse Model

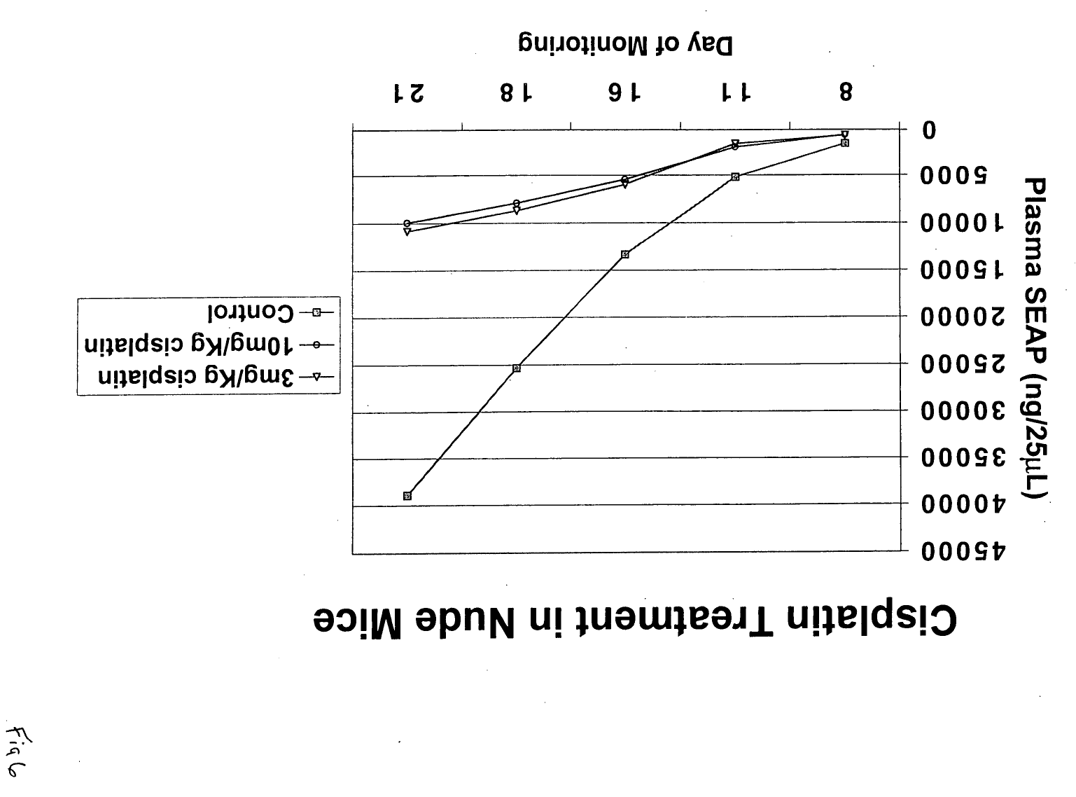

Three nude mice were injected intraperitoneally with 1×10⁷ OCC1-SEAP cells. After 1 week the mice were treated with 3 or 10 mg/kg cisplatin every 48-72 h over a 2-week period or treated with vehicle as controls. Blood samples over this time period were assayed for plasma SEAP. These results shown are representative of three different experiments. Plasma SEAP measurements indicated that cisplatin treatments decreased tumor growth compared to the untreated vehicle controls (FIG. 6). No difference was seen in tumor growth between mice treated with 3 and 10 mg/kg cisplatin.

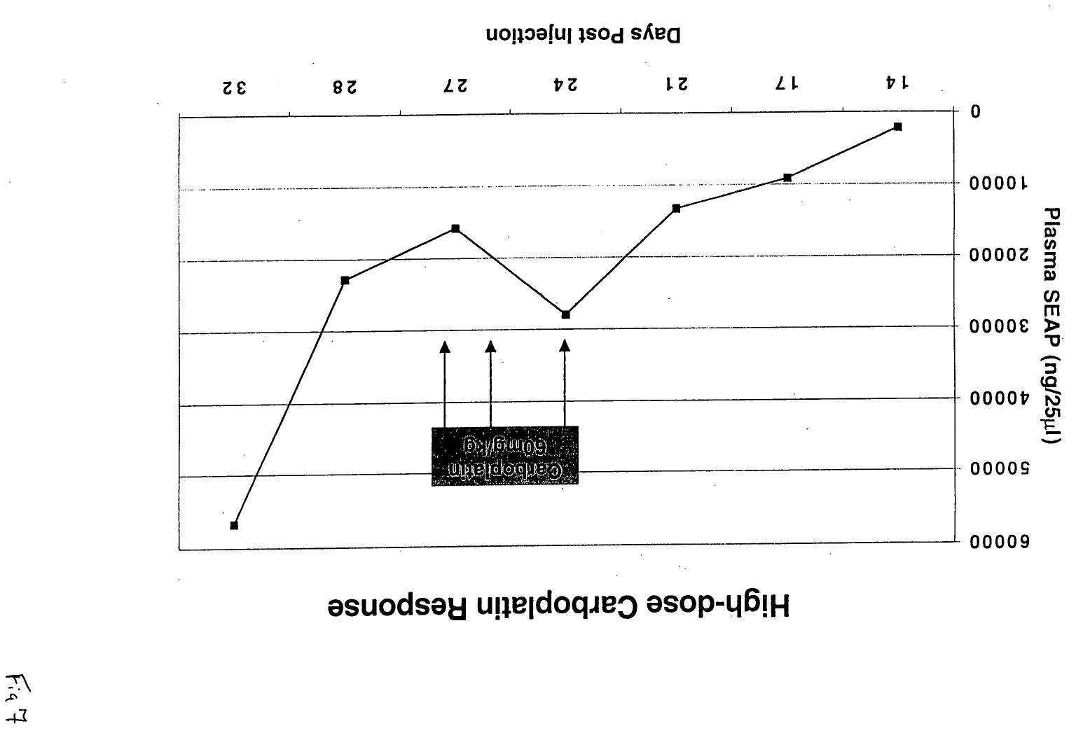

Four nude mice with established OCC1-SEAP intraperitoneal tumors (4 weeks after injection of 1×10⁷ OCC1-SEAP cells) were treated with a high dose of carboplatin (60 mg/kg) three times over a period of 4 days. Blood samples were taken at intervals and assayed for SEAP. The results shown are the response of one mouse and are representative of two different experiments. The arrows indicate when mice were treated with carboplatin. Plasma SEAP levels indicated that carboplatin transiently decreased the tumor burden (FIG. 7). Tumor growth resumed after the treatment was terminated. Therefore, the SEAP tumor model of the invention is effective at monitoring tumor burden and response to platinum-containing chemotherapeutic drugs.

Discussion

In this example, an in vivo tumor model system was used, in which the tumor cell line was transfected with the constitutively expressed marker gene SEAP. The tumor cell line with the marker gene was then grown in nude mice. The SEAP protein is secreted by tumor cells, and can be detected in blood samples. The mice were treated with anticancer chemotherapeutic drugs and the response of the tumors to treatment was evaluated by measuring SEAP levels in the blood over the duration of the experiment.

OCC1-SEAP cells were injected subcutaneously into nude mice to evaluate the accuracy of this model in determining tumor burden. The mice were monitored for tumor growth by measuring the subcutaneous mass through the skin and calculating tumor volume. At the same time plasma SEAP levels were determined. Plasma SEAP was generally found to correlate closely with calculated tumor volume (FIG. 1) in subcutaneous tumors. Interestingly, in a mouse in which the subcutaneous tumor had invaded the underlying body wall, plasma SEAP levels were found to be a better indicator of tumor burden than tumor volume (FIG. 2). SEAP levels would be expected also to be a better indicator of tumor burden than volume for tumors that develop necrotic centers, because only viable tumor cells produce the SEAP protein.

Some tumors, including ovarian carcinomas, arise primarily in the abdominal cavity. For these types of cancers the subcutaneous environment may not be the appropriate system for investigation. OCC1-SEAP cells were injected intraperitoneally into nude mice to evaluate the accuracy of this in vivo model system for determining intraperitoneal tumor burden. At the end of 2-3 weeks of tumor growth the animals were killed and all visible tumor was dissected out and weighed. Blood samples from the time of death were assayed for SEAP levels. Tumor mass was found to correlate with SEAP levels (R²=0.8732; FIG. 3). This correlation may have been improved if during dissection more of the disseminated tumor foci had been located and excised. Also the invasion of tumor cells into the body wall and abdominal organs made isolation of tumor tissue from host tissue problematic. Indeed, it has been shown that some carcinomas will incorporate host stromal tissue into the tumor mass itself (Parrott, Nilsson et al. 2001). Since SEAP levels can be measured over the course of an experiment, tumor burden can be monitored during treatment rather than just at the time of death. These considerations suggest blood SEAP levels are a more accurate and informative indicator of tumor burden than dissecting and measuring tumors at the end-point of the experiment.

One important purpose of a cancer model system is to test the response of tumor cells to anticancer therapeutic drugs. Platinum-containing drugs are used in anticancer therapies for ovarian carcinomas (Council 2000; Misset, Bleiberg et al. 2000; Thigpen 2000). Experiments were performed in vitro and in vivo to test whether SEAP levels reflect the anti-proliferative action of platinum-containing drug treatment. For in vitro experiments, OCC1-SEAP cells were cultured and treated for 2 days with carboplatin. Carboplatin treatment decreased cell proliferation compared to untreated control cells (FIG. 5). This was accompanied by a corresponding decrease in SEAP levels in the culture medium. As discussed above, accurate measurement of intraperitoneal tumor mass is problematic. For this reason, and because repeated measurement of intraperitoneal tumor mass over time in individual mice is extremely difficult, no attempt was made to correlate tumor mass with SEAP levels in these mice. Therefore, SEAP levels are a good indicator of cell number and response to carboplatin treatment in vitro.

Nude mice were injected intraperitoneally with OCC1-SEAP cells and treated with the platinum-containing drug, cisplatin, to see whether the in vitro response also occurred in vivo. Cisplatin-treated mice had lower plasma SEAP levels indicating lower tumor burden than vehicle-treated control mice (FIG. 6). Similarly, when daily SEAP production by OCC1-SEAP cells in vitro was measured, SEAP levels corresponded to cell number on each day in both carboplatin-treated and untreated wells. In another experiment, mice carrying established intraperitoneal tumors were treated with a high dose of carboplatin. Carboplatin-treated mice had a transient decrease in plasma SEAP levels suggesting partial regression of the tumor (FIG. 7). Therefore, this in vivo mouse model system for monitoring ovarian tumor growth reflects responses to platinum-containing therapeutic drugs. In the current study OCC1 ovarian carcinoma cells were shown to respond to platinum-containing chemotherapeutic drugs in vitro and in vivo.

The SEAP secretion by OCC1-SEAP cells was not shown to be regulated independently of cell number. If treatment with a chemotherapeutic drug caused the constitutive levels of SEAP production and secretion to change, the use of SEAP levels as an indicator of tumor burden would be misleading. When SEAP levels were normalized per microgram DNA in in vitro cultures, it was shown that treatment with carboplatin did not change constitutive levels of SEAP secretion in these OCC1 cells (FIG. 5).

In this example, the in vitro response to platinum-containing drugs corresponded to the in vivo animal model. It was also demonstrated that platinum-containing drug treatment did not change the constitutive rate of SEAP secretion per OCC1 cell. These results indicate that SEAP can be used as an in vivo reporter gene mouse model, to monitor tumor growth and response to therapeutic drugs.

### EXAMPLE II

Effects of Combined Treatment Using Triptolide a P13-kinase Inhibitor and Carboplatin, on Ovarian Cancer In Vivo

This example demonstrates the effectiveness of triptolide to inhibit cancer cell survival in vitro, and the ability of triptolide as an effective treatment for ovarian cancer in vivo, either alone, or in combination with other therapeutic treatments.

Materials and Methods

Cell Culture

The human ovarian cancer cell line OCC1 was obtained from Dr. Gordon Mills (MD Anderson Cancer Center, Houston, Tex.) and cultured. The cells were grown in Ham's F-12 medium (Life Technologies), plus 10% bovine calf serum (BCS) as described above in Example I. Once cells reached confluence they were trypsinized and split into appropriate plates.

Cell Survival

Cell survival was assessed as the number of cells remaining in culture, following exposure to treatments. The DNA content of individual culture wells was used as an indication of cell number. Cells plated in 24 well culture plates were allowed to approach confluence (80%) in Ham's F-12 medium plus 10% BCS. Cultures were then incubated in DMEM, plus 0.1% BSA and 0.1% BCS, in the presence of vehicle control, carboplatin (0-100 μg/ml), Triptolide (100 ng/ml) or a combination of these for 24 to 96 hrs. Media aliquots were taken when appropriate for SEAP analysis. The DNA was measured fluorometrically, as described above, in Example I. Briefly, the fluorescence of an aliquot of sonicated cell suspension, into which SYBR Green I fluorescent dye (Molecular Probes, Eugene, Oreg.), had been incorporated was measured.

DNA Isolation and Analysis

Following 48 h to 72 h treatment incubations, cells were suspended into culture medium and pelleted in tubes. DNA was isolated from collected cells using a Puregene™ DNA isolation kit (Gentra Systems, Minneapolis, Minn.). The quantity and purity of nucleic acid preparations were estimated by measuring the absorbance of each sample (A₂₆₀/A₂₈₀). DNA preparations (10 μg/well) were loaded onto 1.2% agarose gels and visualized with ethidium bromide stain.

Western Blot Analysis

The OCC1-SEAP cells were grown to 80% confluence. Cultures were incubated in DMEM plus 0.1% BSA, and 0.1% BSC, to which either vehicle control, carboplatin (0-50 μg/ml), triptolide (100 ng/ml) or combinations of these treatments, had been added. Following 24, 48 or 72 h, cells were lysed with sample buffer (62.5 mM Tris-HCl, pH 6.8, 2% SDS, 5% glycerol, 0.003% glycerol, 0.5% P-mercaptoethanol). Total cell lysates were subjected to SDS-PAGE and transferred to PVDF membranes (Millipore, Bedford, Mass.). Membranes were immunoblotted with antibodies to both cleaved and full length capsase-3 (Cell Signaling Technology, Beverly, Mass.).

Nude Mouse Tumor Model

Athymic nude mice (NCR Nu/Nu) were either purchased from Taconic (Germantown, N.Y.) or bred in-house at Washington State University, Pullman, Wash. The OCC1-SEAP cells were collected in Hank's balanced salt solution and counted prior to injection. Treatments were initiated 7 to 10 days following an intraperitoneal inoculation of mice with 1×10⁷ OCC1-SEAP cells, as described above, in Example I.

Stock solutions of carboplatin (paraplatin; Bristol-Myers Squibb, Princeton, N.J.) and TPL (Calbiochem, Calif.), were prepared in sterile filtered PBS and DMSO, respectively. Animals received intraperitoneal injections of either vehicle controlled (PBS containing 4% DMSO), or carboplatin (60 mg/kg), alone, or in combination with TPL (0-1.0 mg/kg), every other day for 5 days. Alternatively, TPL (0.15 mg/kg) was injected every day for 10 days. For these experiments, carboplatin was administered under the same schedule of every other day for 5 days. Some mice were also treated with 40 mg/kg of LYS294002 every other day for 5 days, with an intraperitoneal injection in the presence of TPL and carboplatin. Blood samples were collected into capillary tubes from saphenous vein lancings at regular intervals during and following treatments. The capillary tubes were centrifuged and the plasma samples were frozen at −20° C., until the time of SEAP assay. The Washington State University Animal Care and Use Committee approved all procedures.

SEAP Assay

Blood plasma and cell culture medium samples were assayed for SEAP activity using the Great EscAPe SEAP fluorescence detection kit (Clontech Laboratories, Palo Alto, Calif.), as described above in Example I. Blood plasma samples were diluted 1:100, prior to the assay to bring values within the linear range of the standard curve. The intraassay and interassay coefficients of variation were 2.5% and 18.8%, respectively. In the case of the experiments in which mice were treated with a combination of TPL, LY294002 and carboplatin, there was some variation in the extent of tumor growth in the control mice. In order to better compare response to treatment across experiments, the SEAP assay data between experiments were normalized by dividing each data point by the overall mean of all SEAP measurements from that experiment and up, through day 18. The normalized data then reflect the relative tumor burden of each mouse, compared to others in the experimental group, and can be combined across experiments.

Statistical Analysis

Data were analyzed by a one-way or two-way analysis of variance (ANOVA). Significant differences between treatment groups were determined using a Student's t-test. In some cases, after analysis of variance, a post-hoc Bonferroni test was used to determine differences between treatment groups at particular time points. For the survival date, a Mantel-Hanszel logrank test (Altman D G. Practical Statistics for Medical Research, ed. 1st. New York: Chapman and Hall, 1991) was used to determine if the survival curves were significantly different. The majority of analyses were performed using Graphpad Prism version 4.0b for MAC (Graphpad Software, San Diego, Calif.). Data are expressed as means ±standard error of the mean (SEM).

Results

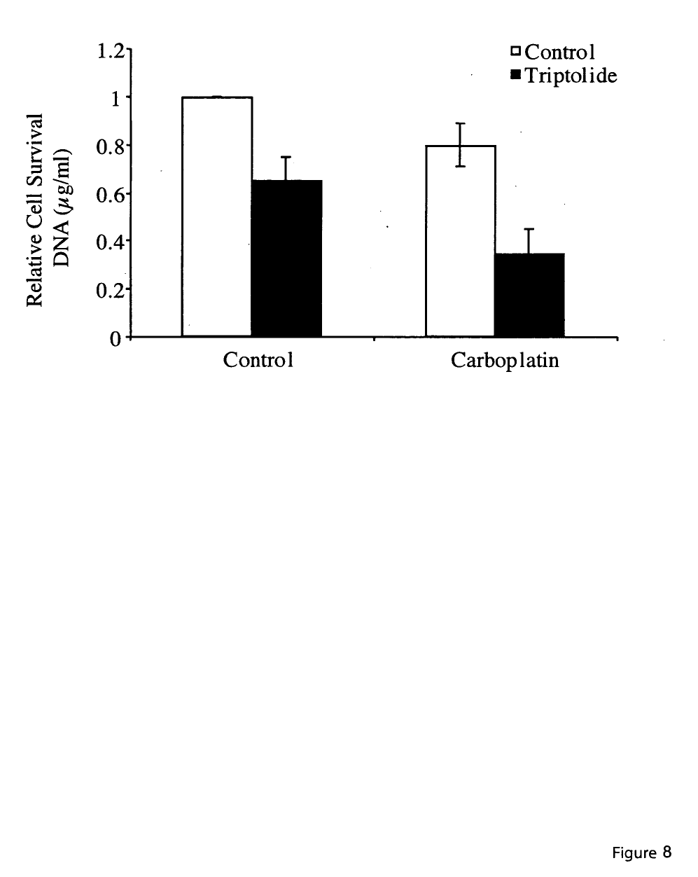

Compared to control cultures, cell survival (i.e. μg DNA/ml) was decreased by approximately 40% in cultures treated with TPL alone, and by 30% in cultures incubated in the presence of carboplatin (FIG. 8). The reduction in cell survival (65%) was greatest in cultures exposed to the combined treatment of TPL and carboplatin.

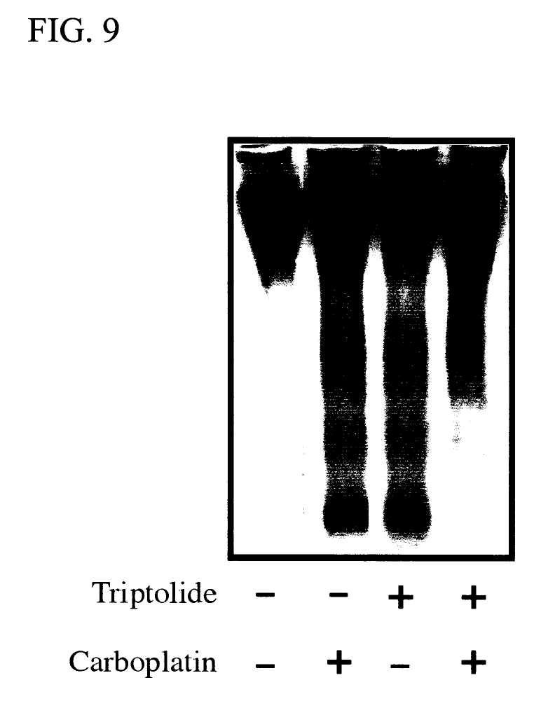

The presence of DNA fragmentation was assessed to determine if reduction in cancer cell number correlated with an increase in cellular apoptosis. The DNA in cells undergoing apoptosis is cleaved by endonucleases, resulting in DNA fragmentation that can be detected electrophoretically (Raff M. Cell suicide for beginners. Nature 1998;396(6707):119-22). DNA laddering was evident in cells after a 48 hr treatment with TPL (100 ng/ml) and carboplatin (50 μg/ml) as well as with the combined treatment of TPL and carboplatin (FIG. 9).

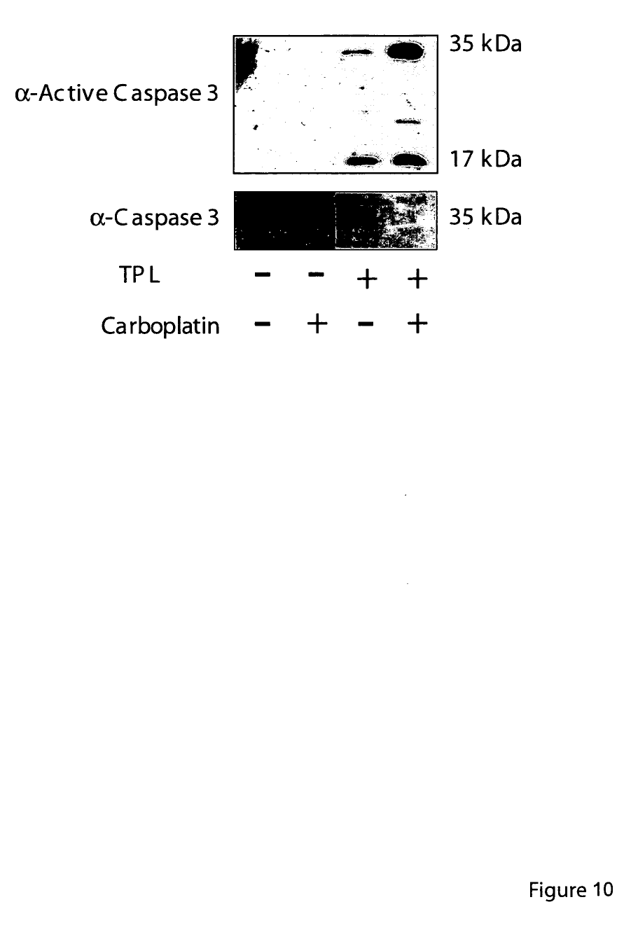

Activation of caspase-3 was used as an additional indicator of apoptosis induction. Caspase-3 is activated downstream of initiator caspase-9 or caspase-8, and is considered the major effector caspase for this proteolytic cascade (Cohen G M. Caspases: the executioners of apoptosis. Biochem J 1997;326 (Pt 1):1-16). Western blot analysis was performed with an antibody that is specific to the active form of caspase-3. A major band of 17 kDa and a minor band of 19 kDa, were visualized with this antibody (FIG. 10). Blots were probed with an antibody to full-length (35 kDa) caspase-3. TPL exposure resulted in robust induction of active caspase-3 at 24 h, and the observed stimulation of caspase-3 was visibly enhanced following combined treatment with both carboplatin and TPL (FIG. 10). Cells treated with carboplatin alone, do not activate caspase-3 until 48 hr post-treatment. Full length caspase-3 protein levels were visually decreased in correlation to the increase in the observed active cleaved products.

Since TPL was found to decrease ovarian cancer cell survival and enhance carboplatin action, in vivo experiments were performed as described in this Example. In initial experiments, a range of doses was used for evaluation of the toxicity of TPL. Once tumors were determined to be established in mice, as indicated by measurable SEAP protein in blood samples, doses of TPL (0.125 mg/kg to 1.0 mg/kg) were administered by intraperitoneal injection every other day over a 5-day period (3 injections). Within 24 hours, 2 of 3 mice receiving the highest dose of 1.0 mg/kg expired. There were not significant differences in SEAP values between mice treated with either vehicle control or any of the remaining doses of TPL (0.125 mg/kg, 0.25 mg/kg or 0.5 mg/kg).

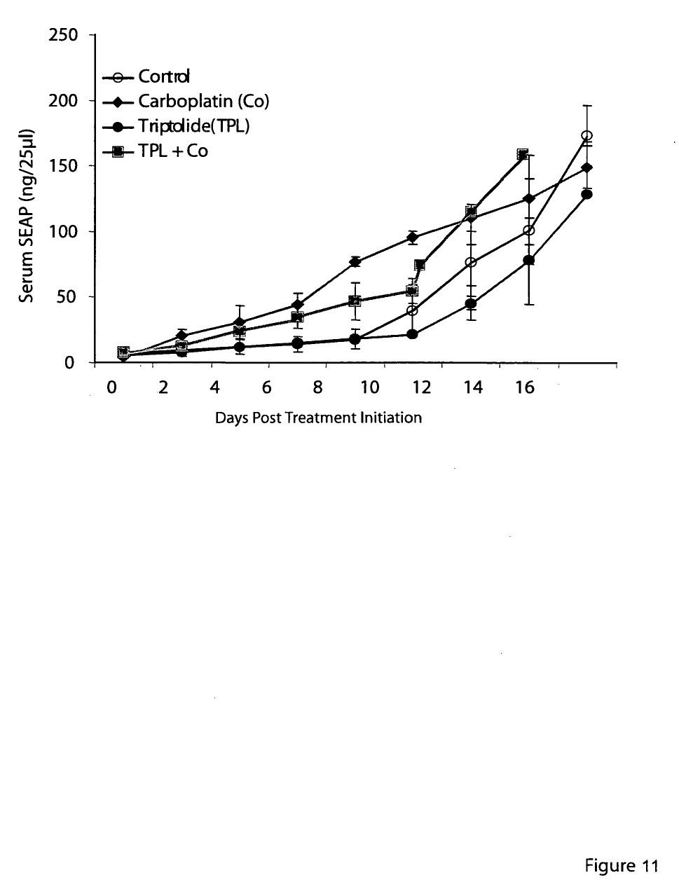

As there was no apparent toxicity associated with the dose of 0.5 mg/kg, this dose was chosen for the following experiment. Carboplatin and TPL were administered separately or in combination, using the same protocol of every other day for 5 days. One of 4 mice receiving the combined treatment of 60 mg/kg carboplatin and 0.5 mg/kg TPL, expired following treatment. No apparent adverse side effects were observed in the remaining mice from this or other treatment groups. There was no significant difference in tumor burden amongst mice receiving vehicle control, TPL, carboplatin or the combined treatment (FIG. 11).

Because no effect was seen with the dose of 0.5 mg/kg triptolide administered over 5 days, the following experiments were conducted with a lower dose of TPL administered over a longer time period. The dose of 0.15 mg/kg has previously been shown to be 60% of the maximum tolerated dose in nude mice, and did not appear to adversely affect the mice (Kiviharju T M, Lecane P S, Sellers R G, Peehl D M. Antiproliferative and proapoptotic activities of triptolide (PG490), a natural product entering clinical trials, on primary cultures of human prostatic epithelial cells. Clin Cancer Res 2002;8(8):2666-74).

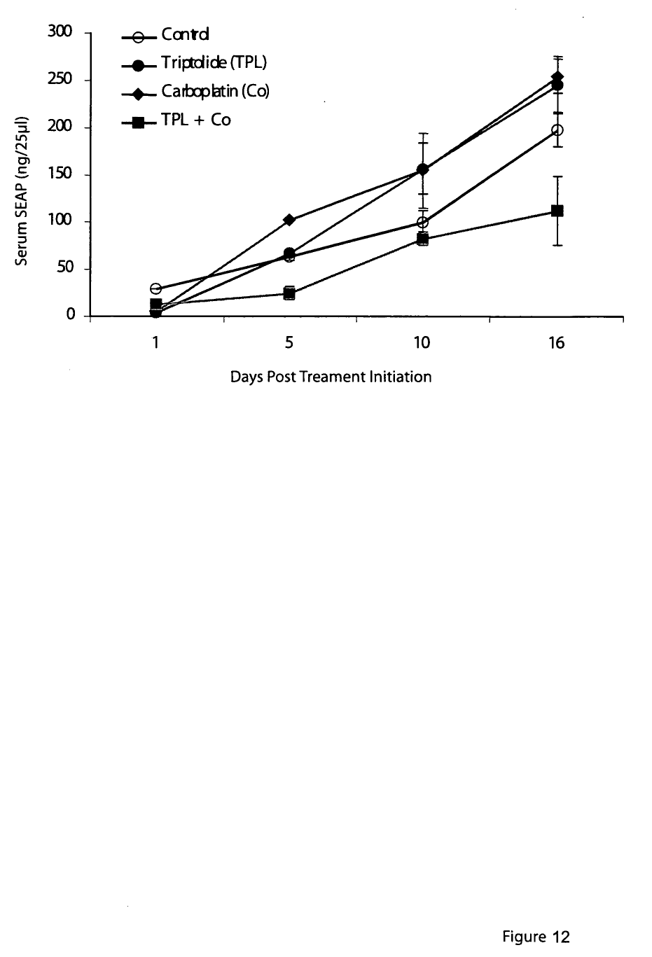

Therefore, the dose of 0.15 mg/kg was chosen for intraperitoneal injection of mice every day for 10 days. The dose and time frame of injection of carboplatin were not altered for these experiments. Mice received their first and last injection of carboplating on the fourth and eight day of TPL administration, respectively. When treatments were administered in this manner, a significant decrease in tumor burden, as measured by SEAP levels, was observed with the combined treatment of TPL and carboplatin (FIG. 12). There was no significant difference in tumor burden between mice receiving TPL or carboplatin alone, and those receiving vehicle control (FIG. 12). There were no adverse side effects observed following this low dose of TPL treatment, or in the combined treatment groups.

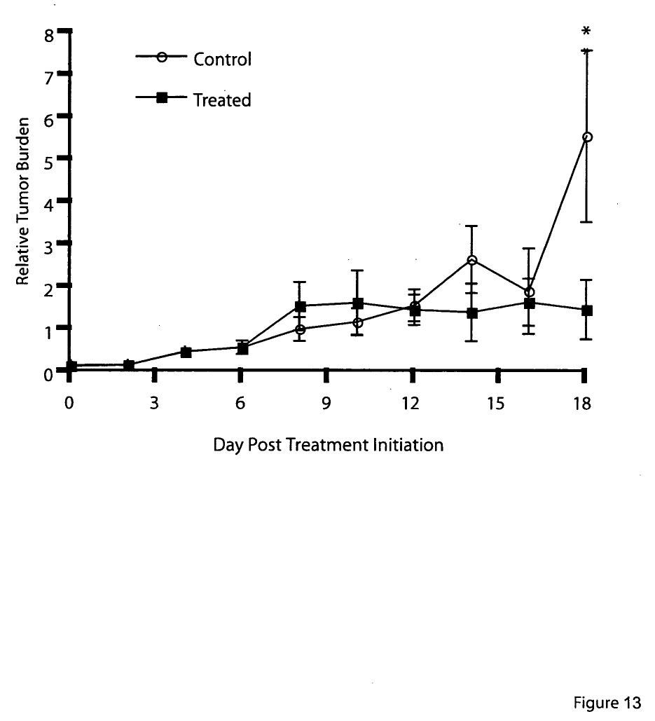

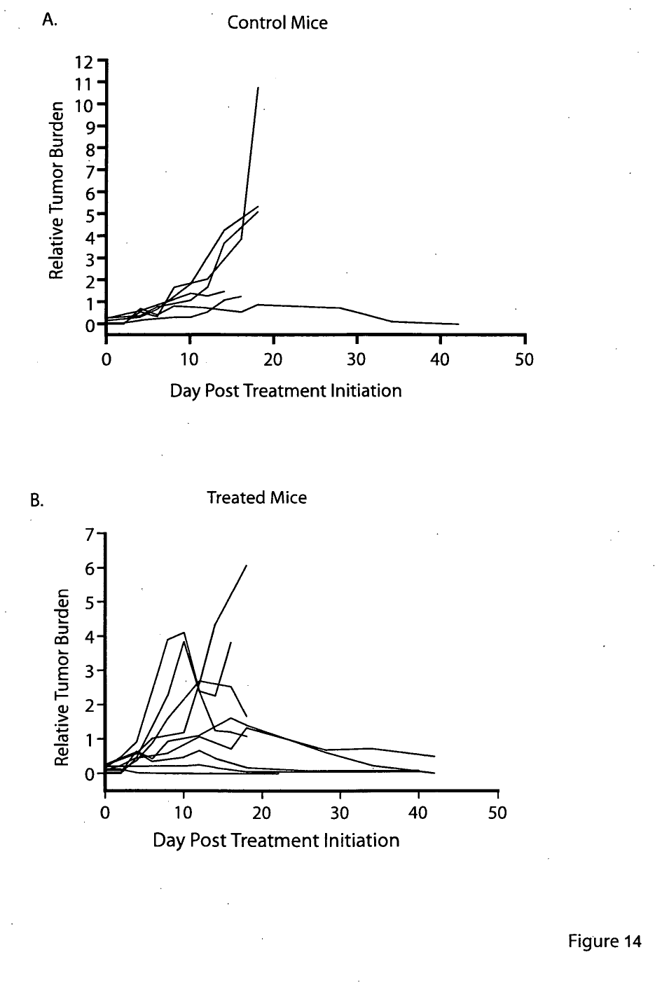

Previously, the P13-kinase inhibitor LY294002, was found to enhance carboplatin actions in vitro and in vivo (Westfall S D, Skinner M K. Inhibition of phosphatidylinositol 3-kinase (PI3K) sensitizes ovarian cancer cells to carboplatin and allows adjunct chemotherapy treatment. Molecular Cancer Therapeutics 2005, 4:1764-71). The potential combined effect of TPL and LY294002 on carboplatin actions was investigated using the nude mouse ovarian tumor model described herein (Nilsson E E, Westfall S D, McDonald C, Ligon T, Sadler-Riggleman I, Skinner M K. An in vivo mouse reporter gene (human secreted alkaline phosphatase) model to monitor ovarian tumor growth and response to therapeutics. Cancer Chemother Pharmacol 2002;49(2):93-100). LY294002 (40 mg/kg) was injected 3 times in 5 days in combination with TPL (0.15 mg/kg daily for 10 days) and carboplatin (60 mg/kg 3 times in 5 days). Alone, Ly294002 had negligible effects on ovarian cancer tumor progression (Westfall S D, Skinner M K. Inhibition of phosphatidylinositol 3-kinase (PI3K) sensitizes ovarian cancer cells to carboplatin and allows adjunct chemotherapy treatment. Molecular Cancer Therapeutics 2005, 4:1764-71). The combined treatment of TPL, LY294002 and carboplatin caused a decrease in ovarian tumor burden and growth, FIG. 13). The tumor burdens of each mouse over time are presented for both those receiving the combination treatment (FIG. 14B), and controls (FIG. 14A). Six of nine treated mice (22%) that were euthanized for ascites and cachexia were found to have had decreasing tumor levels after having developed rather high relative tumor burdens of 304, using normalized SEAP units. 44% (4/9) animals with the combined treatment had total regression of the ovarian tumor as determined by the absence of measurable SEAP levels after 40 days.

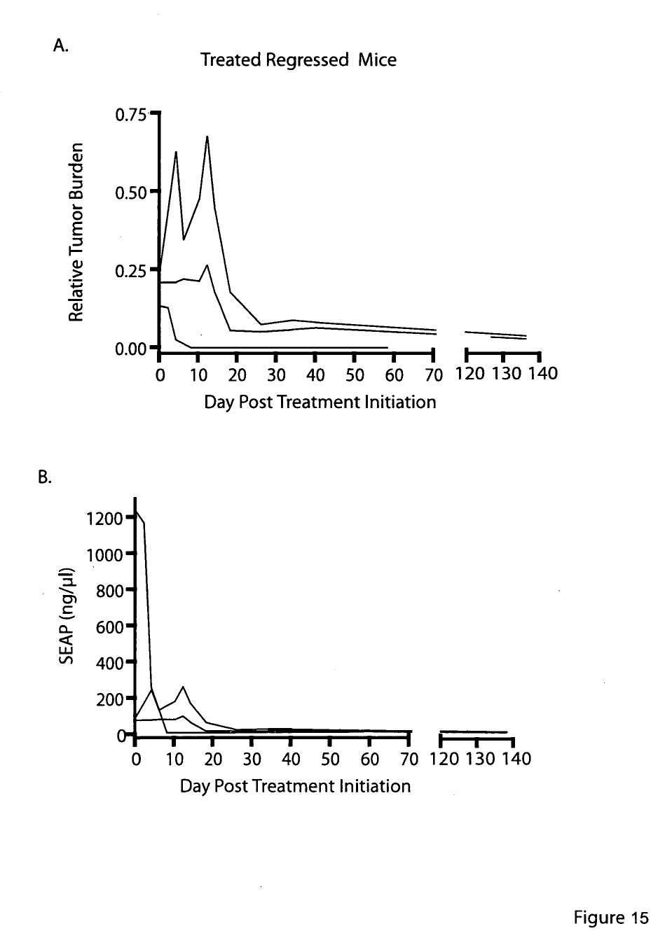

Representative animals are shown in FIG. 15, demonstrating the long-term absence of ovarian cancer. Three surviving mice from the treated group were still tumor-free 2 months after treatment (FIGS. 15A and 15B). One mouse from the control group was able to suppress tumor growth without treatment, although SEAP measurements never fell completely to baseline (FIG. 14A). This mouse was euthanized for ascites production, 42 days after initiation of treatment.

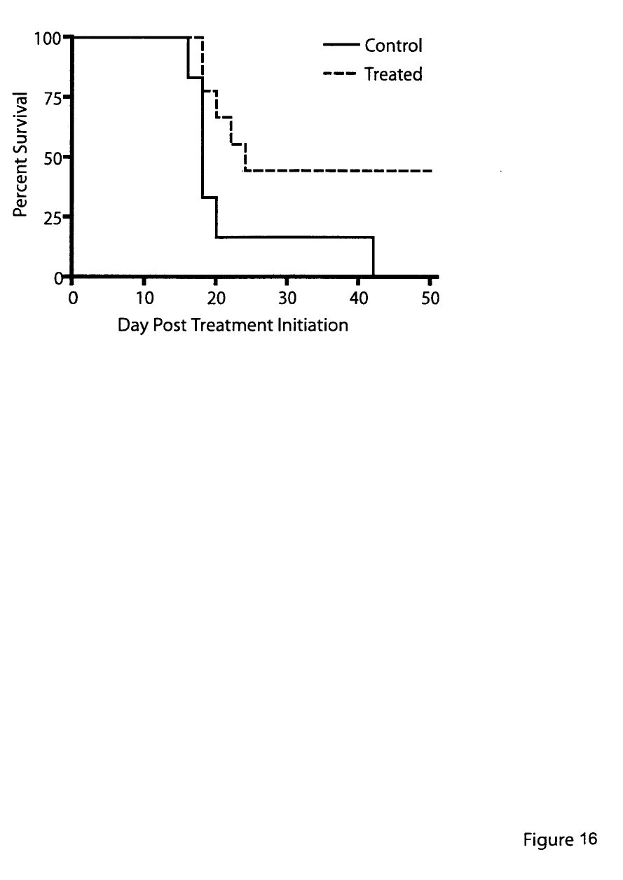

Mice in these studies were euthanized when abdominal distention from ascites fluid or cachexis weight loss reached certain levels, as directed by the approved animal use protocol. Survival curves for the combined TPL, LYS294002 and carboplatin-treated and control mice are shown in FIG. 16. These survival curves were found to be significantly (p<0.05) different, indicating that mice receiving the combined treatment survived significantly longer. Taken together, these studies indicate that TPL did enhance the chemosensitivity of ovarian cancer to carboplatin with the appropriate treatment regimen, but that the optimum effect was observed with combined TPL. LYS294002 and carboplatin treatment.

These results demonstrate that TPL may be used as adjunct chemotherapy for the treatment of ovarian cancer. TPL was a potent stimulator of apoptosis in OCC1-SEAP cell cultures, as indicated by DNA fragmentation and induction of caspase-3 activity. The observed increase in apoptosis correlated with a decrease in cell number. The combined treatment of TPL and carboplatin further increased DNA laddering and caspase-3 activity, suggesting that TPL enhances the ability of carboplatin to induce OCC1-SEAP cell death in vitro. TPL also enhanced the cytotoxicity of carboplatin in vivo. There was not an observable increase in the effects of carboplatin when TPL was used at a high dose over a short time period. However, when administered at a low dose on a daily basis, TP increased the ability of carboplatin to inhibit OCC1-SEAP tumor growth in nude mice.

The observed results may be due to a specific range of efficacy for TPL. In the current experiment, a dose of 1.0 mg/kg TPL was lethal. Half of this dose (0.5 mg/kg) was not effective in reducing tumor burden alone, or in conjunction with carboplatin and had toxic effects when administered with carboplatin. A study of Shamon et al. found the dose of 0.5 mg/kg to be lethal in athymic mice with breast cancer xenografts (Shamon L A, Pezzuto J M, Graves J M, Mehta RR , Wangcharoentrakul S, Sangsuwan R, et al. Evaluation of the mutagenic, cytotoxic, and antitumor potential of triptolide, a highly oxygenated diterpene isolated from Tripterygium wilfordii. Cancer Lett 1997;112(1):113-7). Because of the toxicity observed with the dose of 0.5 mg/kg, further experiments were conducted using a lower dose of 0.15 mg/kg TPL. In addition, the period of injection was increased from every other day for 5 days to every day for 10 days. The dose of 0.15 mg/kg was demonstrated to be potent in inhibiting growth and metastasis of several solid tumor types in nude mice studies conducted by Yang et al. (Yang S, Chen J, Guo Z, Xu X M, Wang L, Pei X F, et al. Triptolide inhibits the growth and metastasis of solid tumors. Mol Cancer Ther 2003;2(1):65-72). In the current example, TPL was not effective as a single agent, but did exhibit the ability to increase the efficacy of carboplatin in this system. The fact that TPL is not thought to influence the PI3-kinase/Akt signal transduction pathway that LY294002 inhibits, with the present results suggest that combined block of PI3-kinase by LY294002 and action of TPL to partially inhibit P53 and NκB are required to obtain an optimal adjunct chemotherapy.

These results demonstrate the effectiveness of TPL in inhibiting ovarian cancer tumor growth and survival, either as a single chemotherapy agent, or in combination with platinum based therapy. PL enhanced the cytotoxicity of carboplatin in culture and enhanced carboplatin-mediated reduction of tumor burden in nude mice inoculated with human ovarian cancer cells. The combined treatment of TPL, PI3 kinase inhibitor LY294002 and carboplatin, was found to dramatically reduce ovarian tumor progression and burden in nude mice. In 44% of the animals tested the combined treatment caused complete regression of ovarian cancer. Combined observations indicate TPL suppresses chemoresistance to carboplatin, and is an effective adjunct chemotherapy for ovarian cancer.

### EXAMPLE III

Effects of Treatment Using a P13-kinase Inhibitor and Carboplatin, on Ovarian Cancer Cells

This Example examines the ability of a PI 3-kinase inhibitor to render ovarian cancer cells susceptible to the effects of platinum-based chemotherapy.

Materials and Methods

Cell Culture

Human ovarian cancer cell line OCC1 were modified to constitutively express the SEAP gene as described above in Example I. The OCC1 cells were stably transfected with Fugene™ reagent with a pCMV-SEAP plasmid. The clonal isolate that produced high levels of SEAP (OCC1-SEAP-12) was used in subsequent in vitro and in vivo experiments. The OCC1-SEAP-12 cells were grown in HAM's F-12 medium (Life Technologies) plus 10% bovine calf serum (BCS). Once cells reached confluence they were trypsinized and sub-cultured into appropriate plates.

Growth Assays

Cell proliferation was analyzed by determining the amount of [³H] thymidine incorporation Into newly synthesized DNA. The OCC1-SEAP-12 cells were plated in 24 well plates in Ham's F-12 medium plus 10% BCS and allowed to reach 50 to 70% confluence. Following a 48 hr serum starvation, the culture medium was changed to Dulbecco's Modified Eagle Medium (DMEM) plus 0.1% BSA and 0.1% BCS containing either vehicle control or carboplatin (0-100 μg/ml) alone, or in combination with LY294002 (0-20 μM). Treatments were removed after 18 h and cells were incubated for 4 hr in medium containing 5 μCi/ml of [³H] thymidine. Medium was removed and cells were disrupted by sonication in phosphate buffered saline (PBS). An aliquot of the sonicated solution of PBS was loaded onto a DEAE filtration plate (Millipore, Bedford, Mass.), and individual filters with bound DNA were collected for scintillation counting. Data was normalized to total DNA per well and was determined by a SYBR green fluorescent assay (Nilsson E E, Westfall S D, McDonald C, Ligon T, Sadler-Riggleman I, Skinner M K. An in vivo mouse reporter gene (human secreted alkaline phosphatase) model to monitor ovarian tumor growth and response to therapeutics. Cancer Chemother Pharmacol 2002;49(2):93-100.).

Cell Survival

Cell survival was assessed as cell number remaining in culture following exposure to treatments. The DNA content of individual culture wells was used as an indication of cell number. Cells plated in 24 well culture plates were allowed to approach confluence (80%) in Ham's F-12 medium plus 10% BCS. Cultures were then incubated in DMEM plus 0.1% BSA and 0.1% BCS in the presence of vehicle control, carboplatin (0-100 μg/ml), LY294002 (0-20 μM), or a combination of these for 24 to 96 hr. Media aliquots were taken when appropriate for SEAP analysis. The DNA was measured fluorometrically as previously described (Nilsson E E, Westfall S D, McDonald C, Ligon T, Sadler-Riggleman I, Skinner M K. An in vivo mouse reporter gene (human secreted alkaline phosphatase) model to monitor ovarian tumor growth and response to therapeutics. Cancer Chemother Pharmacol 2002;49(2):93-100.). Briefly, the fluorescence of aliquots of sonicated cell suspensions into which SYBR Green I fluorescent dye (Molecular Probes, Eugene Oreg.) had been incorporated, was measured.

DNA Isolation and Analysis

Following 48 hr to 72 hr treatment incubations, cells were suspended into culture medium and pelleted in tubes. DNA was isolated from collected cells using a Puregene™ DNA isolation kit (Gentra Systems, Minneapolis, Minn.). The quantity and purity of nucleic acid preparations were estimated by measuring the optical density of each sample A₂₆₀/A₂₈₀). DNA preparations (10 μg/well) were loaded onto 1.2% agarose gels and visualized with ethidium bromide stain.

Western Blot Analysis

The OCC1-SEAP-12 cells were grown to 80% confluence. Cultures were incubated in DMEM plus 0.1% bovine serum albumin (BSA) and 0.1% BCS to which either vehicle control, carboplatin (0-50 μg/ml), LYS294002 (0-20 μM) or combinations of these treatments. Following 24, 48 or 72 hr cells were lysed with sample buffer (62.5 mM Tris-HCl, pH 6.8, 2% SDS, 5% glycerol, 0.003% glycerol, 0.5% β-mercaptoethanol). Total cell lysates were subjected to SDS polyacrylamide gel electrophoresis 9 page) and transferred to PVDF membranes (Millipore, Bedford, Mass.). Membranes were immunoblotted with antibodies to both the phosphorylated and non-phosphorylated forms of Akt (Cell Signaling Technology, Beverly, Mass.), or alternatively, with antibodies to cleaved and full length caspase-3 (Cell Signaling Technology, Beverly, Mass.).

Nude Mouse Tumor Model:

Athymic nude mice were obtained and inoculated intraperitoneally with OCC1-SEAP-12 cells as described above in Examples I and II. Animals received injections of either vehicle control (PBS containing 25% DMSO), or carboplatin (60 mg/kg) alone, or in combination with LY294002 (50 mg/kg) every other day for 6 days. Blood samples were collected into capillary tubes from saphenous vein lancings at regular intervals during and following treatments. Capillary tubes were centrifuged, and the plasma samples were frozen (−20° C.), until the time of SEAP assay. The Washington State University Animal Care and Use Committee approved all procedures.

SEAP Assay

Blood plasma and cell culture medium samples were assayed for SEAP activity using the Great EscAPe SEAP fluorescence detection kit (Clontech Laboratories, Palo Alto, Calif.) as described above in Examples I and II. Blood plasma samples were diluted 1:100 prior to the assay to bring values within the linear range of the standard curve. The intraassay and interassay coefficients of variation were 2.5% and 18.8%, respectively.

Statistical Analysis

Data were analyzed by a one-way analysis of variance (ANOVA). Significant differences between treatment groups were determined using a student's t-test. Data were expressed as means ±standard error of the mean (SEM).

Results

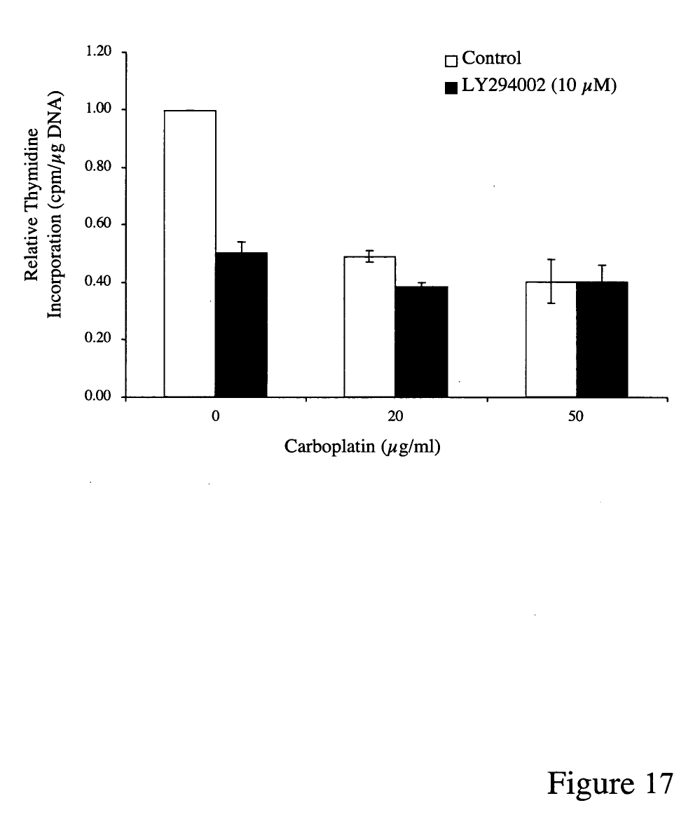

Ovarian cancer cells characteristically grow independent of growth factor stimulation. Basal levels of DNA synthesis of OCC1-SEAP-12 cells were blocked in cultures incubated in the presence of LY294002 by 50%, and by 51% and 59.5%, following treatment with 20 μg/ml and 50 μg/ml carboplatin, respectively (FIG. 17). The combined treatment of LY294002 and 20 μg/ml carboplatin did not reduce DNA synthesis more than either treatment alone (FIG. 17). The reduction in thymidine incorporation was not significantly different than that seen following treatment with 50 μg/ml carboplatin alone, or in combination with LY294002 (FIG. 17). Observations demonstrate that PI 3-kinase inhibition results in decreased OCC1-SEAP-12 thymidine incorporation to the same degree as seen with the chemotherapeutic agent, carboplatin.

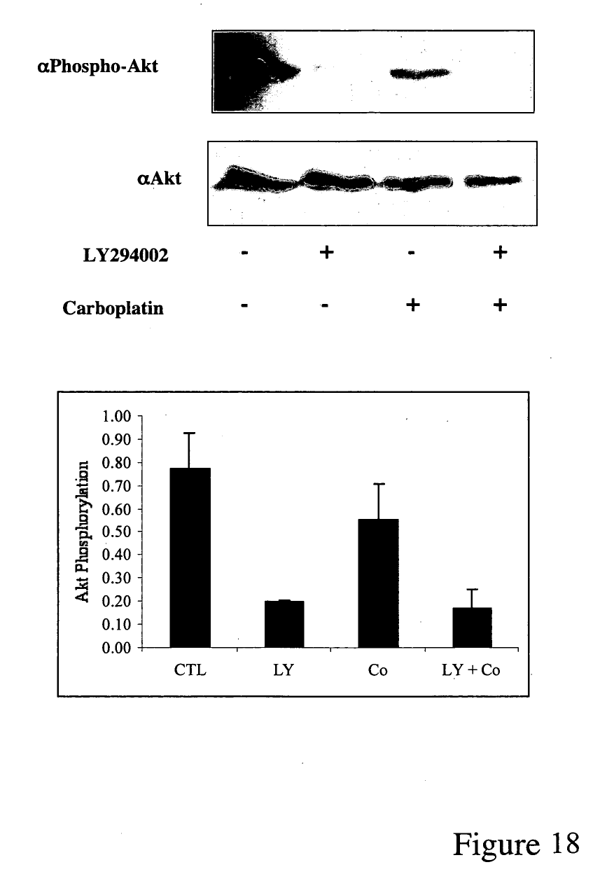

Western blot analysis was employed to determine if stimulation of the PI 3-kinase pathway was blocked in OCC1-SEAP-12 cell cultures by LY294002. Total cell lysates from OCC1-SEAP-12 cultures treated with LY294002 and/or carboplatin were subjected to SDS-PAGE, transferred to nylon membrane and probed with an antibody specific to the phosphorylated form of Akt. Basal levels of Akt phosphorylation were observed in lysates from unstimulated OCC1-SEAP-12 cell cultures (FIG. 18). It has been reported that cislatin treatment stimulates activation of the PI 3-kinase pathway (Hayakawa J, Ohmichi M, Kurachi H, et al. Inhibition of BAD phosphorylation either at serine 112 via extracellular signal-regulated protein kinase cascade or at serine 136 via Akt cascade sensitizes human ovarian cancer cells to cisplatin. Cancer Res 2000;60(21):5988-94). Carboplatin had not effect on Akt phosphorylation at either 6 hr, or 24 hr in OCC1-EAP-12 cells (FIG. 18). The PI 3-kinase inhibitor, LY294002 blocked basal levels of Akt phosphorylation at 6 hr and 24 hr (FIG. 18). The inhibition of Akt phosphorylation by LY294002 was not affected by the presence of carboplatin. Inhibition of basal levels of Akt activity correlated with inhibition of basal levels of DNA synthesis.

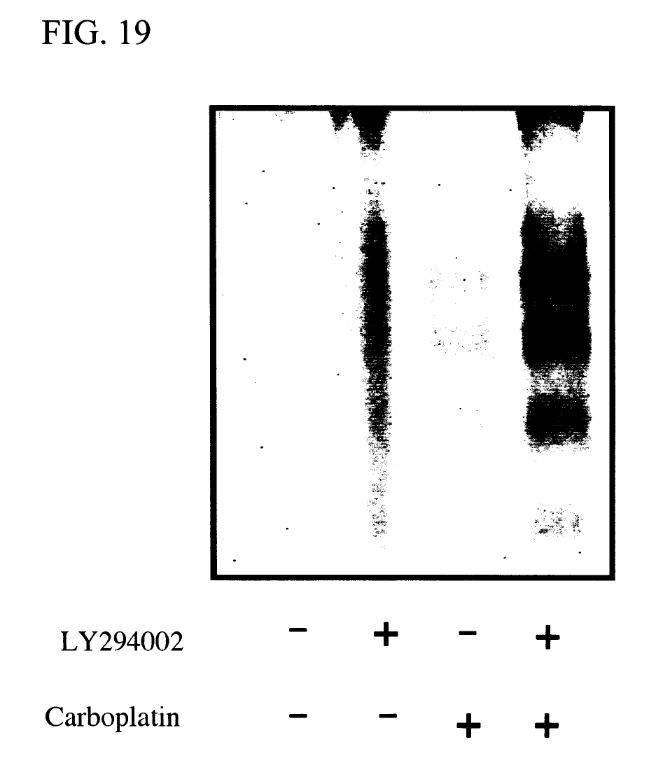

Cell cultures were assessed for the presence of apoptosis following treatment with either LY294002 or carboplatin. The DNA in cells undergoing apoptosis is cleaved by endonucleases resulting in DNA fragmentation that can be detected electrophoretically (Raff M. Cell suicide for beginners. Nature 1998; 396(6707):119-22). There was no DNA laddering evident in samples from untreated control cultures at either 24 hr or 72 hr (FIG. 19). DNA laddering was observed in cultures of OCC1-SEAP-12 cells incubated in the presence of carboplatin with and without LY294002 at 48 hr and 72 hr (FIG. 19). An increase in DNA laddering was evident with the combined LY294002 and carboplatin treatment (FIG. 19).

Caspases are proteolytic enzymes that play a central role in the regulation of apoptosis and are activated prior to apoptotic DNA degradation (Cohen G M. Caspases: the executioners of apoptosis. Biochem J 1997;326 (Pt 1):1-16). Caspases are expressed as an inactive precursor and are activated in an amplifying proteolytic cascade (Cohen G M. Caspases: the executioners of apoptosis. Biochem J 1997;326 (Pt 1):1-16). Among the caspases, caspase 3 is considered to be a major executioner protease (Cohen G M. Caspases: the executioners of apoptosis. Biochem J 1997;326 (Pt 1):1-16). Western blot analysis was used to determine the amount of the activated caspase-3 present.

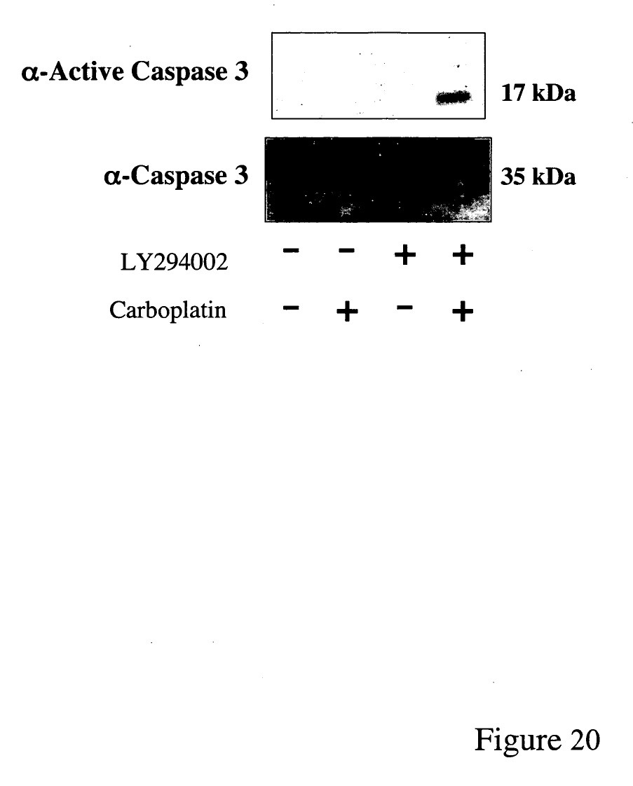

Procaspase-3 is expressed as a 33 kDa protein and is cleaved into 17 kDa and 12 kDa proteolytic products (Cohen G M. Caspases: the executioners of apoptosis. Biochem J 1997;326 (Pt 1):1-16). The active 17 kDa caspase 3 was evident at 24 hr in cells treated with the combination of carboplatin and LY294002 (FIG. 20). In contrast, the active form of caspase 3 did not appear until 48 hr in cultures treated with carboplatin alone and not until 72 hr in cultures treated with LY294002 alone. There was a concomitant loss of full length caspase-3 in these samples (FIG. 20).

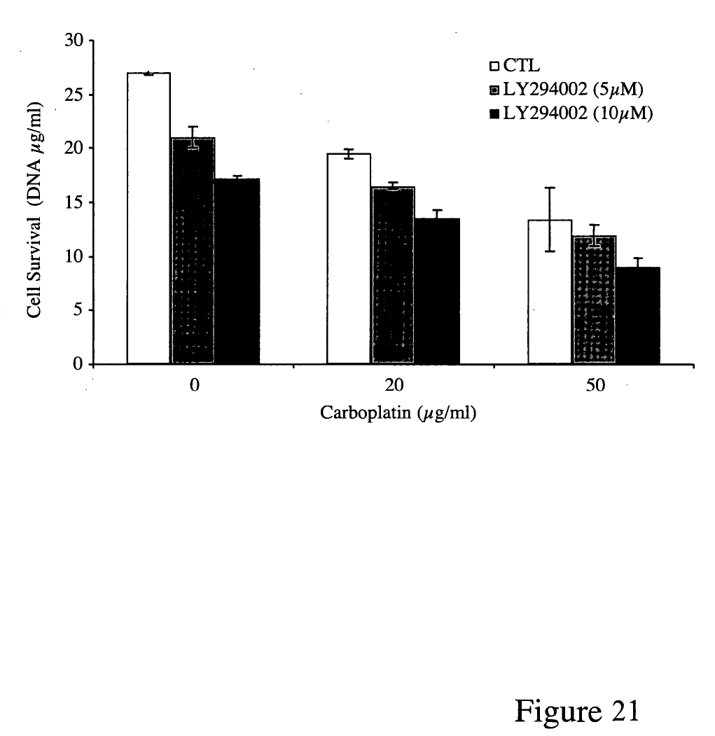

To determine if the observed increase in apoptosis resulted in a decrease in cell survival, the cell number remaining in culture following prolonged exposure to carboplatin and/or LY294002 was assessed. Levels of DNA in each culture well following 48 hr incubations in the absence or presence of LY294002 with or without carboplatin, were used as an indicator of cell number. Doses of 20 μg/ml and 50 μg/ml carboplatin were used for these experiments as they correlated with in vivo doses used. Two doses of LY294002 (5 μM and 10 μM), were chosen as optimal from the performed dose response curve. At these concentrations, LY294002 has been shown to specifically inhibit PI 3-kinase and is within the effective dose range shown to inhibit PI 3-kinase activity in a variety of cell types. After cells in culture had reached near confluence, treatments were added to culture media. Following a 48 h incubation, cell number was reduced by 22% and 44% by 5 μM and 10 μM LY294002, respectively (FIG. 21). There was no significant difference in cell number remaining following the combined treatment of 20 μg/ml carboplatin and 10 μM LY294002, and 50 μg/ml carboplatin alone (FIG. 21). Less than half the amount of carboplatin in combination with LY294002 reduced cell survival to the same extent as the high dose of carboplatin (FIG. 21). Observations indicate that the combined treatment with LY294002 and carboplatin induced optimal apoptosis in the ovarian cancer cells.

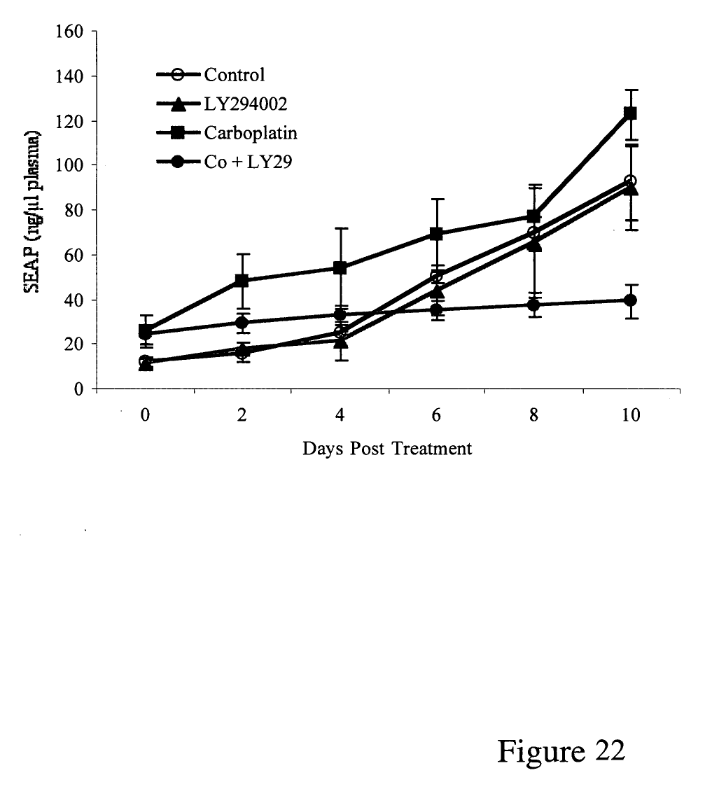

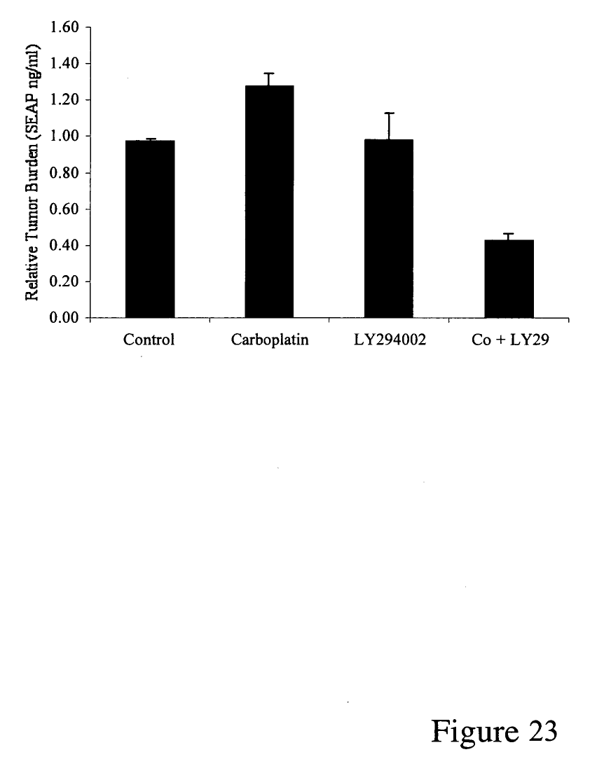

In vivo studies were initiated to extend the in vitro assessment of the ability of LY294002 to inhibit ovarian cancer cell growth and cell survival. Nude mice were given intraperitoneal injection of OCC1-SEAP-12 cells, and plasma levels of SEAP were used to assay tumor establishment, one week following OCC1 cell inoculation (Nilsson E E, Westfall S D, McDonald C, Ligon T, Sadler-Riggleman I, Skinner M K. An in vivo mouse reporter gene (human secreted alkaline phosphatase) model to monitor ovarian tumor growth and response to therapeutics. Cancer Chemother Pharmacol 2002;49(2):93-100). Mice were then injected with vehicle control, carboplatin and/or LY294002, every other day for 6 days. The SEAP levels were monitored during and following the treatment regimen. Tumors in mice receiving the combined treatment of LY294002 (50 mg/kg) and carboplatin (60 mg/kg), had a suppressed growth curve, when compared to tumors in mice that were treated with vehicle control or LY294002 or carboplatin alone (FIG. 22). Tumors in these mice eventually approached the size of tumors in mice from other treatment groups, however, tumor growth was significantly retarded. In all experiments, ascites formation was found to parallel tumor growth. Mice receiving carboplatin treatment alone, exhibited muscle wasting and became anemic. The drug induced toxicity was not observed with combined treatment. At the point at which tumor burden and ascites formation caused excessive abdominal swelling and/or mice displayed toxic side-effects, they were euthanized. Using these parameters, mice receiving combined treatment lived much longer. Of the 6 mice receiving the combined treatment of carboplatin and LY294002 in FIG. 23, 4 lived 57% longer than the mice in the remaining treatment groups and 1 mouse lived 43% longer.

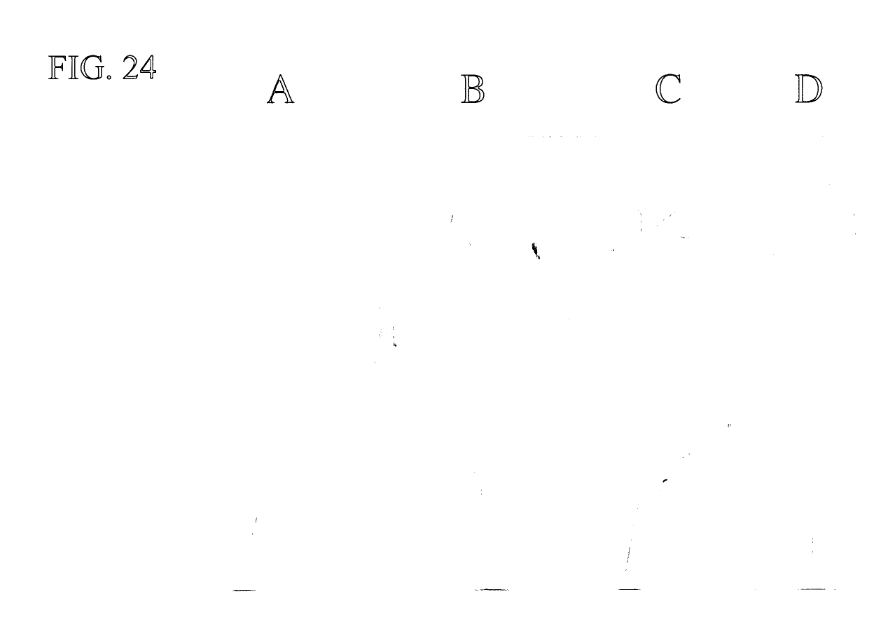

When comparing SEAP levels of the final common bleed for all mice, tumor size was decreased by 2.3 fold as a result of combined treatment of LY294002 and carboplatin, in comparison to vehicle control (FIG. 23). There was no significant difference in tumor size amongst vehicle control, LY294002 and carboplatin treatment groups (FIG. 23). The reduction in tumor burden and ascites formation was also evident in the physical appearance of mice (FIG. 24). Mice from vehicle control, LY294002 and carboplatin treatment groups displayed abdominal swelling that is characteristic of ascites formation and excessive tumor burden (FIG. 24). There was a significant reduction in abdominal swelling in mice treated with the combination of LY294002 and carboplatin (FIG. 24). During the experiment, 2 mice treated with the combined treatment of carboplatin and LY294002 exhibited complete remission with no measure SEAP, 30 days past cessation of treatment. These mice were sacrificed at this point, and had no observable tumor. However, complete remission was not observed in any other mice (n=10), following carboplatin and LY294002 treatment, and therefore, these mice with complete remission were not included in final averages.

These results demonstrate that inhibition of the PI 3-kinase/Akt pathway results in a decreased proliferation of OCC1-SEAP-12 cells in vitro. LY294002 blocked Akt phosphorylation in OCC11-SEAP-12 cultures. The reduction in levels of phosphorylated Akt correlated with the inhibition of proliferation. These and other previous observations, demonstrate that the normal mitogenic response of cancer cells can be overcome by inhibiting the PI 3-kinase/Akt signaling pathway. Additionally, the present experiment corresponds to a recent report by Gao et al., which demonstrated that LY294002 inhibition of PI 3-kinase resulted in G1 cell cycle arrest in ovarian cancer cells which corresponded to the up-regulation of p16^{INK4A} expression (Gao N, Flynn D C, Zhang Z, et al. G1 cell cycle progression and the expression of G1 cyclins are regulated by PI3K/AKT/mTOR/p70S6K1 signaling in human ovarian cancer cells. Am J Physiol Cell Physiol 2004;287(2):C281-91). Cell cycle progression following exposure to DNA damaging agents, such as platinum based compounds, is blocked by p53 activation and subsequent p21^{CIP1/WAF1} expression (Ferreira C G, Epping M, Kruyt F A, Giaccone G. Apoptosis: target of cancer therapy. Clin Cancer Res 2002;8(7):2024-34). Despite contrasting mechanisms, LY294002 and carboplatin were equally effective, but not additive, in blocking ovarian cancer cell proliferation in this experiment.

In addition to the attenuation of OCC1-SEAP-12 cell proliferation, a decrease in cell survival was seen following PI 3-kinase inhibition. However, compared to the growth response, LY294002 alone, was not as effective in promoting apoptosis as carboplatin. The combination of both compounds was additive, as indicated by a marked enhancement of DNA laddering in cells, following the combined treatment of carboplatin and LY294002. Furthermore, activation of caspase 3 was induced at a much earlier time point with the combined treatment. The active or cleaved form of caspase 3 was evident within 24 hours following combined treatment and was not detectable until 48 h following carboplatin treatment alone, or at 72 hr, following LY294002 treatment alone. Also, a significantly low dose of carboplatin was needed to reduce cell number in culture when in the presence of LY294002. Observations suggest that inhibition of the PI 3-kinase/Akt pathway can sensitize ovarian cancer cells to the toxic effects of carboplatin.

These results demonstrate that LY294002 in combination with carboplatin was effective in inhibiting ovarian cancer cell xenograft growth in a nude mouse model. There was a significant delay in growth of ovarian tumors in mice receiving both carboplatin and LY294002, compared to other treatment groups. This resulted in a significant increase in survival rate when compared to all other treatment groups. The current study demonstrated LY294002 alone, was note effective in inhibiting tumor progression as was observed in a previous study by Hu et al. (Hu L, Zaloudek C, Mills G B, Gray J, Jaffe R B. In vivo and in vitro ovarian carcinoma growth inhibition by a phosphatidylinositol 3-kinase inhibitor (LY294002). Clin Cancer Res 2000;6(3):880-6; Hu L, Hofmann J, Lu Y, Mills G B, Jaffe R B. Inhibition of phosphatidylinositol 3′-kinase increases efficacy of paclitaxel in in vitro and in vivo ovarian cancer models. Cancer Res 2002;62(4):1087-92). This is most likely a result of use of a dose of 50 mg/kg LY294002, versus a dose of 100 mg/kg, which was found to be most effective in the experiments performed by Hu et al. In addition, the number of injections was fewer, and the duration of treatment was shorter (every other day for 5 days versus 3 days a week for 4 weeks). The current experiment focused on the potential use of PI 3-kinase inhibitor as an adjunct chemotherapy with carboplatin.

The combined treatment of carboplatin and LY294002 inhibits ovarian tumor progression, and supports the use of the PI 3-kinase inhibitor, LY294002, with the platinum-based drug therapy, as an appropriate treatment course for ovarian cancer.

Because the compound LY294002 is toxic when given systemically, its administration must be intraperitoneally have to be through intraperitoneal infusion. Ovarian cancer generally remains confined to the abdominal cavity, and as a result, intraperitoneal infusion of therapy is an appropriate delivery system for this disease, making LY294002 a plausible alternative chemotherapeutic agent.

### REFERENCES

Altman D G. Practical Statistics for Medical Research, ed. 1st. New York: Chapman and Hall, 1991.
Altomare D A, Wang H Q, Skele K L, et al. AKT and mTOR phosphorylation is frequently detected in ovarian cancer and can be targeted to disrupt ovarian tumor cell growth. Oncogene 2004;23(34):5853-7.
American Cancer Society (1998) Cancer facts and figures. American Cancer Society, Atlanta.
Anderson, P. (1997). “Kinase cascades regulating entry into apoptosis.” Microbiol Mol Biol Rev 61(1): 33-46.
Arcaro, A. and M. P. Wymann (1993). “Wortmannin is a potent phosphatidylinositol 3-kinase inhibitor: the role of phosphatidylinositol 3,4,5-trisphosphate in neutrophil responses.” Biochem J 296 (Pt 2): 297-301.
Auersperg N, Maines-Bandiera S L, Dyck H G. Ovarian carcinogenesis and the biology of ovarian surface epithelium. J Cell Physiol 1997;173(2):261-5.
Auersperg N, Edelson M I, Mok S C, Johnson S W, Hamilton T C. The biology of ovarian cancer. Semin Oncol 1998;25(3):281-304.
Baldwin, A. S. (2001). “Control of oncogenesis and cancer therapy resistance by the transcription factor NF-kappaB.” J Clin Invest 107(3): 241-6.
Bao, R., M. Selvakumaran, et al. (2000). “Use of a surrogate marker (human secreted alkaline phosphatase) to monitor in vivo tumor growth and anticancer drug efficacy in ovarian cancer xenografts.” Gynecol Oncol 78(3 Pt 1): 373-9.
Bassendine M, Arborgh B, Shipton U, Monjardino J, Aranguibel F, Thomas H, Sherlock S (1980) Hepatitis B surface antigen and alpha-fetoprotein secreting human primary liver cell cancer in athymic mice. Gastroenterology 79:528-532.
Berger, J., J. Hauber, et al. (1988). “Secreted placental alkaline phosphatase: a powerful new quantitative indicator of gene expression in eukaryotic cells.” Gene 66(1): 1-10.
Blagosklonny M V. Prospective strategies to enforce selectively cell death in cancer cells Oncogene 2004;23(16):2967-75.
Blumenthal, R. D., E. Leone, et al. (2004). “An in vitro model to optimize dose scheduling of multimodal radioimmunotherapy and chemotherapy: effects of p53 expression.” Int J Cancer 108(2): 293-300.
Carpenter, C. L. and L. C. Cantley (1990). “Phosphoinositide kinases.” Biochemistrv 29(51): 11147-56.
Carpenter, C. L., B. C. Duckworth, et al. (1990). “Purification and characterization of phosphoinositide 3-kinase from rat liver.” J Biol Chem 265(32): 19704-11.
Carpenter C L, Cantley L C. Phosphoinositide 3-kinase and the regulation of cell growth. Biochim Biophys Acta 1996;1288(1):M11-6.
Casagrande, F., D. Bacqueville, et al. (1998). “G1 phase arrest by the phosphatidylinositol 3-kinase inhibitor LY 294002 is correlated to up-regulation of p27Kip1 and inhibition of G1 CDKs in choroidal melanoma cells.” FEBS Lett 422(3): 385-90.
Chan, E. W., S. C. Cheng, et al. (2001). “Triptolide induced cytotoxic effects on human promyelocytic leukemia, T cell lymphoma and human hepatocellular carcinoma cell lines.” Toxicol Lett 122(1): 81-7.
Chang, W. T., J. J. Kang, et al. (2001). “Triptolide and chemotherapy cooperate in tumor cell apoptosis. A role for the p53 pathway.” J Biol Chem 276(3): 2221-7.
Chen, B. J. (2001). “Triptolide, a novel immunosuppressive and anti-inflammatory agent purified from a Chinese herb Tripterygium wilfordii Hook F.” Leuk Lymphoma 42(3): 253-65.
Cheng J Q, Jiang X, Fraser M, et al. Role of X-linked inhibitor of apoptosis protein in chemoresistance in ovarian cancer: possible involvement of the phosphoinositide-3 kinase/Akt pathway. Drug Resist Updat 2002;5(3-4):131-46.
Chmura, S. J., E. Nodzenski, et al. (1997). “Loss of ceramide production confers resistance to radiation-induced apoptosis.” Cancer Res 57(7): 1270-5.
Cohen G M. Caspases: the executioners of apoptosis. Biochem J 1997;326 (Pt 1): 1-16.
Council, M. R. (2000). “Chemotherapy for advanced ovarian cancer-Advanced ovarian cancer trialists group.” MRC Clinical Trials Unit, Meta-Analysis Group, Cochrane Library System Review 2(cd001418).
Crul, M., J. H. Schellens, et al. (1997). “Cisplatin resistance and DNA repair.” Cancer Treat Rev 23(5-6): 341-66.
Datta, S. R., H. Dudek, et al. (1997). “Akt phosphorylation of BAD couples survival signals to the cell-intrinsic death machinery.” Cell 91(2): 231-41.
Davis, R. J. (1993). “The mitogen-activated protein kinase signal transduction pathway.” J Biol Chem 268(20): 14553-6.
del Peso, L., M. Gonzalez-Garcia, et al. (1997). “Interleukin-3-induced phosphorylation of BAD through the protein kinase Akt.” Science 278(5338): 687-9.
Delmastro, D. A., J. Li, et al. (1997). “DNA damage inducible-gene expression following platinum treatment in human ovarian carcinoma cell lines.” Cancer Chemother Pharmacol 39(3): 245-53.
Dudek, H., S. R. Datta, et al. (1997). “Regulation of neuronal survival by the serine-threonine protein kinase Akt.” Science 275(5300): 661-5.
Eastman, A. (1990). “Activation of programmed cell death by anticancer agents: cisplatin as a model system.” Cancer Cells 2(8-9): 275-80.
Eastman, A. (1995). “Survival factors, intracellular signal transduction, and the activation of endonucleases in apoptosis.” Semin Cancer Biol 6(1): 45-52.
Ferguson, P. J. (1995). “Mechanisms of resistance of human tumours to anticancer drugs of the platinum family: a review.” J Otolaryngol 24(4): 242-52.
Ferreira, C. G., C. Tolis, et al. (1999). “p53 and chemosensitivity.” Ann Oncol 10(9): 1011-21.
Ferreira C G, Epping M, Kruyt F A, Giaccone G. Apoptosis: target of cancer therapy. Clin Cancer Res 2002;8(7):2024-34.
Fidler, J. M., K. Li, et al. (2003). “PG490-88, a derivative of triptolide, causes tumor regression and sensitizes tumors to chemotherapy.” Mol Cancer Ther 2(9): 855-62.
Fingl, E. and D. M. Woodbury (1975). General Principles. The Pharmacological basis of therapeutics. L. S. Goodman and A. Gilman. New York, Macmillan: 1.
Fraser M, Leung B, Jahani-Asl A, Yan X, Thompson W E, Tsang B K. Chemoresistance in human ovarian cancer: the role of apoptotic regulators. Reprod Biol Endocrinol 2003;1(1):66.22
Fresno Vara J A, Casado E, de Castro J, Cejas P, Belda-Iniesta C, Gonzalez-Baron M. PI3K/Akt signalling pathway and cancer. Cancer Treat Rev 2004;30(2):193-204. 20
Gao N, Flynn D C, Zhang Z, et al. G1 cell cycle progression and the expression of G1 cyclins are regulated by PI3K/AKT/mTOR/p70S6K1 signaling in human ovarian cancer cells. Am J Physiol Cell Physiol 2004;287(2):C281-91.
Gosland, M., B. Lum, et al. (1996). “Insights into mechanisms of cisplatin resistance and potential for its clinical reversal.” Pharmacotherapy 16(1): 16-39.
Grasso, A. W., D. Wen, et al. (1997). “ErbB kinases and NDF signaling in human prostate cancer cells.” Oncogene 15(22): 2705-16.
Hautkappe A, Lu M, Mueller H, Bex A, Harstrick A, Roggendorf M, Ruebben H (2000) Detection of germ-cell tumor cells in the peripheral blood by nested reverse transcriptase-polymerase chain reaction for alpha-fetoprotein-messenger RNA and beta huan chorionic gonadotropin-messenger RNA. Cancer Res 60:3170-3174.
Hayakawa, J., M. Ohmichi, et al. (2000). “Inhibition of BAD phosphorylation either at serine 112 via extracellular signal-regulated protein kinase cascade or at serine 136 via Akt cascade sensitizes human ovarian cancer cells to cisplatin.” Cancer Res 60(21): 5988-94.
Herrin, V. E. and J. T. Thigpen (1999). “Chemotherapy for ovarian cancer: current concepts.” Semin Surg Oncol 17(3): 181-8.
Hu, L., C. Zaloudek, et al. (2000). “In vivo and in vitro ovarian carcinoma growth inhibition by a phosphatidylinositol 3-kinase inhibitor (LY294002).” Clin Cancer Res 6(3): 880-6.
Hu, Q., A. Klippel, et al. (1995). “Ras-dependent induction of cellular responses by constitutively active phosphatidylinositol-3 kinase.” Science 268(5207): 100-2.
Hu L, Hofmann J, Lu Y, Mills G B, Jaffe R B. Inhibition of phosphatidylinositol 3′-kinase increases efficacy of paclitaxel in in vitro and in vivo ovarian cancer models. Cancer Res 2002;62(4):1087-92.
Jiang, X. H., B. C. Wong, et al. (2001). “Functional p53 is required for triptolide-induced apoptosis and AP-1 and nuclear factor-kappaB activation in gastric cancer cells.” Oncogene 20(55): 8009-18.
Kasid, U., S. Suy, et al. (1996). “Activation of Raf by ionizing radiation.” Nature 382(6594): 813-6.
Katabuchi H, Okamura H. Cell biology of human ovarian surface epithelial cells and ovarian carcinogenesis. Med Electron Microsc 2003;36(2):74-86.
Kauffmann-Zeh, A., P. Rodriguez-Viciana, et al. (1997). “Suppression of c-Myc-induced apoptosis by Ras signalling through PI(3)K and PKB.” Nature 385(6616): 544-8.
Kennedy, S. G., A. J. Wagner, et al. (1997). “The PI 3-kinase/Akt signaling pathway delivers an anti-apoptotic signal.” Genes Dev 11(6): 701-13.
Khwaja, A., P. Rodriguez-Viciana, et al. (1997). “Matrix adhesion and Ras transformation both activate a phosphoinositide 3-OH kinase and protein kinase B/Akt cellular survival pathway.” Embo J 16(10): 2783-93.
Khwaja A. Akt is more than just a Bad kinase. Nature 1999;401(6748):33-4.
Kim D, Dan H C, Park S, Yang L, Liu Q, Kaneko S, et al. AKT/PKB signaling mechanisms in cancer and chemoresistance. Front Biosci 2005;10:975-87.
Kiviharju, T. M., P. S. Lecane, et al. (2002). “Antiproliferative and proapoptotic activities of triptolide (PG490), a natural product entering clinical trials, on primary cultures of human prostatic epithelial cells.” Clin Cancer Res 8(8): 2666-74.
Krystal G W, Sulanke G, Litz J. Inhibition of phosphatidylinositol 3-kinase-Akt signaling blocks growth, promotes apoptosis, and enhances sensitivity of small cell lung cancer cells to chemotherapy. Mol Cancer Ther 2002;1(11):913-22.
Kulik, G., A. Klippel, et al. (1997). “Antiapoptotic signalling by the insulin-like growth factor I receptor, phosphatidylinositol 3-kinase, and Akt.” Mol Cell Biol 17(3): 1595-606.
Kyriakis, J. M. and J. Avruch (1996). “Protein kinase cascades activated by stress and inflammatory cytokines.” Bioessays 18(7): 567-77.
Lee, K. Y., W. Chang, et al. (1999). “PG490 (triptolide) cooperates with tumor necrosis factor-alpha to induce apoptosis in tumor cells.” J Biol Chem 274(19): 13451-5.
Lee, K. Y., J. S. Park, et al. (2002). “Triptolide sensitizes lung cancer cells to TNF-related apoptosis-inducing ligand (TRAIL)-induced apoptosis by inhibition of NF-kappaB activation.” Exp Mol Med 34(6): 462-8.
Lin, N., T. Sato, et al. (2001). “Triptolide, a novel diterpenoid triepoxide from Tripterygium wilfordii Hook. f., suppresses the production and gene expression of pro-matrix metalloproteinases 1 and 3 and augments those of tissue inhibitors of metalloproteinases 1 and 2 in human synovial fibroblasts.” Arthritis Rheum 44(9): 2193-200.
Lu, L. H., Y. Y. Lian, et al. (1992). “Clinical Study of Triptolide Treatment of Acute Leukemia.” Clin Exp Investing Hematol 3: 1-3.
Lue, Y., A. P. Sinha Hikim, et al. (1998). “Triptolide: a potential male contraceptive.” J Androl 19(4): 479-86.
Mabuchi, S., M. Ohmichi, et al. (2004). “Inhibition of NFkappaB increases the efficacy of cisplatin in in vitro and in vivo ovarian cancer models.” J Biol Chem 279(22): 23477-85.
Malaguarnera L. Implications of apoptosis regulators in tumorigenesis. Cancer Metastasis Rev 2004;23(3-4):367-87.
Markman, M. (2000). “Weekly paclitaxel in the management of ovarian cancer.” Semin Oncol 27(3 Suppl 7): 37-40.
Marsden D E, Friedlander M, Hacker N F. Current management of epithelial ovarian carcinoma: a review. Semin Surg Oncol 2000;19(1):11-9.
McCormick F. Cancer: survival pathways meet their end. Nature 2004;428(6980):267-9.
McGuire W P, 3rd, Markman M. Primary ovarian cancer chemotherapy: current standards of care. Br J Cancer 2003;89 Suppl 3:S3-8.
McIntire K, Vogel C, Princler G, Patel I (1972) Serum alpha-fetoprotein as a biochemical marker for hepatocellular carcinoma. Cancer Res 32:1941-1946.
Minshall, C., S. Arkins, et al. (1996). “Requirement for phosphatidylinositol 3′-kinase to protect hemopoietic progenitors against apoptosis depends upon the extracellular survival factor.” J Immunol 156(3): 939-47.
Misset, J. L., H. Bleiberg, et al. (2000). “Oxaliplatin clinical activity: a review.” Crit Rev Oncol Hematol 35(2): 75-93.
Mitsuuchi, Y., S. W. Johnson, et al. (2000). “The phosphatidylinositol 3-kinase/AKT signal transduction pathway plays a critical role in the expression of p21WAF1/CIP1/SDI1 induced by cisplatin and paclitaxel.” Cancer Res 60(19): 5390-4.
Nehme, A., R. Baskaran, et al. (1997). “Differential induction of c-Jun NH2-terminal kinase and c-Abl kinase in DNA mismatch repair-proficient and -deficient cells exposed to cisplatin.” Cancer Res 57(15): 3253-7.
Nicosia S V, Bai W, Cheng J Q, Coppola D, Kruk P A. Oncogenic pathways implicated in ovarian epithelial cancer. Hematol Oncol Clin North Am 2003; 17(4):927-43.
Nilsson, E. E., S. D. Westfall, et al. (2002). “An in vivo mouse reporter gene (human secreted alkaline phosphatase) model to monitor ovarian tumor growth and response to therapeutics.” Cancer Chemother Pharmacol 49(2): 93-100.
Ozols, R. F. (1992). “Ovarian cancer, Part II: Treatment.” Curr Probl Cancer 16(2): 61-126.
Ozols, R. F. (2000). “Paclitaxel (Taxol)/carboplatin combination chemotherapy in the treatment of advanced ovarian cancer.” Semin Oncol 27(3 Suppl 7): 3-7.
Ozols R F, Bookman M A, Connolly D C, et al. Focus on epithelial ovarian cancer. Cancer Cell 2004;5(1):19-24.
Parrott, J. A., E. Nilsson, et al. (2001). “Stromal-epithelial interactions in the progression of ovarian cancer: influence and source of tumor stromal cells.” Mol Cell Endocrinol 175(1-2): 29-39.
Parrott, J. A. and M. K. Skinner (1999). “Kit-ligand/stem cell factor induces primordial follicle development and initiates folliculogenesis.” Endocrinology 140(9): 4262-71.
Pignataro, O. P. and M. Ascoli (1990). “Epidermal growth factor increases the labeling of phosphatidylinositol 3,4-bisphosphate in MA-10 Leydig tumor cells.” J Biol Chem 265(3): 1718-23.
Piver, M. S., T. R. Baker, et al. (1991). “Epidemiology and etiology of ovarian cancer.” Semin Oncol 18(3): 177-85.
Qiu D, Zhao G, Aoki Y, Shi L, Uyei A, Nazarian S, et al. Immunosuppressant PG490 (triptolide) inhibits T-cell interleukin-2 expression at the level of purine-box/nuclear factor of activated T-cells and NF-kappaB transcriptional activation. J Biol Chem 1999;274(19):13443-50.
Qiu, D. and P. N. Kao (2003). “Immunosuppressive and anti-inflammatory mechanisms of triptolide, the principal active diterpenoid from the Chinese medicinal herb Tripterygium wilfordii Hook. f.” Drugs R D 4(1): 1-18.
Raff M. Cell suicide for beginners. Nature 1998;396(6707):119-22.
J Reedijk 1987 Pure Appl Chem 59:181-192
Ridderstrale, M. and H. Tomqvist (1994). “PI-3-kinase inhibitor Wortmannin blocks the insulin-like effects of growth hormone in isolated rat adipocytes.” Biochem Biophys Res Commun 203(1): 306-10.
Rosette, C. and M. Karin (1996). “Ultraviolet light and osmotic stress: activation of the JNK cascade through multiple growth factor and cytokine receptors.” Science 274(5290): 1194-7.
Roymans D, Slegers H. Phosphatidylinositol 3-kinases in tumor progression. Eur J Biochem 2001;268(3):487-98.
Sadowski, H. B., K. Shuai, et al. (1993). “A common nuclear signal transduction pathway activated by growth factor and cytokine receptors.” Science 261(5129): 1739-44.
Santana, P., L. A. Pena, et al. (1996). “Acid sphingomyelinase-deficient human lymphoblasts and mice are defective in radiation-induced apoptosis.” Cell 86(2): 189-99.
Schlessinger, J. (1994). “SH2/SH3 signaling proteins.” Curr Opin Genet Dev 4(1): 25-30.
Schuijer M, Bems E M. TP53 and ovarian cancer. Hum Mutat 2003;21(3):285-91.
See H T, Kavanagh J J, Hu W, Bast R C. Targeted therapy for epithelial ovarian cancer: current status and future prospects. Int J Gynecol Cancer 2003;13(6):701-34.
Seger, R. and E. G. Krebs (1995). “The MAPK signaling cascade.” Faseb J 9(9): 726-35.
Shamon, L. A., J. M. Pezzuto, et al. (1997). “Evaluation of the mutagenic, cytotoxic, and antitumor potential of triptolide, a highly oxygenated diterpene isolated from Tripterygium wilfordii.” Cancer Lett 112(1): 113-7.
Shayesteh, L., Y. Lu, et al. (1999). “PIK3CA is implicated as an oncogene in ovarian cancer.” Nat Genet 21(1): 99-102.
Shepherd, J. E. (2000). “Current strategies for prevention, detection, and treatment of ovarian cancer.” J Am Pharm Assoc (Wash) 40(3): 392-401.
Simon, C., M. J. Hicks, et al. (1999). “PD 098059, an inhibitor of ERK1 activation, attenuates the in vivo invasiveness of head and neck squamous cell carcinoma.” Br J Cancer 80(9): 1412-9.
Skinner H D, Zheng J Z, Fang J, Agani F, Jiang B H. Vascular endothelial growth factor transcriptional activation is mediated by hypoxia-inducible factor lalpha, HDM2, and p70S6K1 in response to phosphatidylinositol 3-kinase/AKT signaling. J Biol Chem 2004;279(44):45643-51.
Spencer K (2000)Second-trimester prenatal screening for Down syndrome and the relationship of maternal serum biochemical markers to pregnancy complications with adverse outcome, Prenat Diagn 20:652-656.
Stamey T, Yang N, Hay A, McNeal J, Freiha F, Rewine E (1987) Prostate-specific antigen as a serum marker for adeno-carcinoma of the prostate. N Engl J Med 317:909-916.
Talapatra S, Thompson C B. Growth factor signaling in cell survival: implications for cancer treatment. J Pharmacol Exp Ther 2001;298(3):873-8.
Talmadge C, Tanio Y, Meeker A, Talmadge J, Zbar B (1987) Tumor cells transfected with the neomycin resistance gene (neo) contain unique genetic markers useful for identification of tumor recurrence and metastasis. Invasion Metastasis 7:197-207.
Thant, A. A., A. Nawa, et al. (2000). “Fibronectin activates matrix metalloproteinase-9 secretion via the MEK1-MAPK and the PI3K-Akt pathways in ovarian cancer cells.” Clin Exp Metastasis 18(5): 423-8.
Thigpen, J. T. (2000). “Chemotherapy for advanced ovarian cancer: overview of randomized trials.” Semin Oncol 27(3 Suppl 7): 11-6.
Thomas, J. E., M. Venugopalan, et al. (1997). “Inhibition of MG-63 cell proliferation and PDGF-stimulated cellular processes by inhibitors of phosphatidylinositol 3-kinase.” J Cell Biochem 64(2): 182-95.
Treisman, R. (1996). “Regulation of transcription by MAP kinase cascades.” Curr Opin Cell Biol 8(2): 205-15.
Varticovski, L., D. Harrison-Findik, et al. (1994). “Role of PI 3-kinase in mitogenesis.” Biochim Biophys Acta 1226(1): 1-11.
Vasey, P. A. (2003). “Resistance to chemotherapy in advanced ovarian cancer: mechanisms and current strategies.” Br J Cancer 89 Suppl 3: S23-8.
Vergouwe Y, Steyerberg E, Foster R, Habbema J, Donohue J (2001) Validation of a prediction model and its predictors for the histology of residual masses in non-seminomatous testicular cancer. J Urol 165:84-88.
Verheij, M., R. Bose, et al. (1996). “Requirement for ceramide-initiated SAPK/JNK signalling in stress-induced apoptosis.” Nature 380(6569): 75-9.
Vlahos, C. J., W. F. Matter, et al. (1994). “A specific inhibitor of phosphatidylinositol 3-kinase, 2-(4-morpholinyl)-8-phenyl-4H-1-benzopyran-4-one (LY294002).” J Biol Chem 269(7): 5241-8.
Westfall S D, Skinner M K. Inhibition of phosphatidylinositol 3-kinase (PI3K) sensitizes ovarian cancer cells to carboplatin and allows adjunct chemotherapy treatment. Cancer Chemotherapy and Pharmacology 2005:(Submitted).
Wetzker R, Rommel C. Phosphoinositide 3-kinases as targets for therapeutic intervention. Curr Pharm Des 2004;10(16):1915-22.
Wobst, A., R. A. Audisio, et al. (1998). “Oesophageal cancer treatment: studies, strategies and facts.” Ann Oncol 9(9): 951-62.
Wong, W. S., Y. F. Wong, et. al. (1990). “Establishment and characterization of a new human cell line derived from ovarian clear cell carcinoma.” Gynecol Oncol 38(1): 37-45.
Xia, Z., M. Dickens, et al. (1995). “Opposing effects of ERK and JNK-p38 MAP kinases on apoptosis.” Science 270(5240): 1326-31.
Xu Q, Simpson S E, Scialla T J, Bagg A, Carroll M. Survival of acute myeloid leukemia cells requires PI3 kinase activation. Blood 2003;102(3):972-80.
Yang, S., J. Chen, et al. (2003). “Triptolide inhibits the growth and metastasis of solid tumors.” Mol Cancer Ther 2(1): 65-72.
Yang, Y., Z. Liu, et al. (1998). “Triptolide induces apoptotic death of T lymphocyte.” Immunopharmacology 40(2): 139-49.
Yao, R. and G. M. Cooper (1995). “Requirement for phosphatidylinositol-3 kinase in the prevention of apoptosis by nerve growth factor.” Science 267(5206): 2003-6.
Yao, R. and G. M. Cooper (1996). “Growth factor-dependent survival of rodent fibroblasts requires phosphatidylinositol 3-kinase but is independent of pp70S6K activity.” Oncogene 13(2): 343-51.
Yuan Z Q, Sun M, Feldman R I, Wang G, Ma X, Jiang C, et al. Frequent activation of AKT2 and induction of apoptosis by inhibition of phosphoinositide-3-OH kinase/Akt pathway in human ovarian cancer. Oncogene 2000;19(19):2324-30.
Zhang, T. M., Z. Y. Chen, et al. (1981). “[Antineoplastic action of triptolide and its effect on the immunologic functions in mice (author's transl)].” Zhongguo Yao Li Xue Bao 2(2): 128-31.
Zhang L, Hellstrom K, Chen L (1994) Luciferase activity as a marker of tumor burden and as an indicator of tumor response to antineoplastic therapy in vivo. Clin Exp Metastasis 12:87-92.
Zhang V, Vaillancourt P, Shih I-M, Vogelstein B (2000)New method for quantifying tumor mass in living animals. Stratagene http://www.stratagene.com/vol14_{—}1p21-213/asp.
Zhang L, Yang N, Katsaros D, et al. The oncogene phosphatidylinositol 3′-kinase catalytic subunit alpha promotes angiogenesis via vascular endothelial growth factor in ovarian carcinoma. Cancer Res 2003;63(14):4225-31.
Zhao G, Vaszar L T, Qiu D, Shi L, Kao P N. Anti-inflammatory effects of triptolide in human bronchial epithelial cells. Am J Physiol Lung Cell Mol Physiol 2000;279(5):L958-66.

What is claimed:

1. A pharmaceutical composition, comprising a suitable carrier, and an inhibitor of a PI3K signal transduction pathway, and at least one compound selected from the group consisting of platinum-containing drugs, Taxol or Taxol derivatives, cyclophosphamide, Triptolide, and an antagonist of lysophosphatidic acid (LPA).

2. The composition of claim 1, wherein said platinum-containing drugs are selected from the group consisting of cisplatin, carboplatin and oxaliplatin.

3. A method for inhibiting tumor growth, comprising administering to a subject a therapeutically effective amount of the pharmaceutical composition of claim 1.

4. The method of claim 3, wherein the tumor growth inhibited is ovarian tumor growth.

5. A method for killing tumor cells, comprising administering to a subject a therapeutically effective amount of the pharmaceutical composition of claim 1, sufficient to kill the tumor cells.

6. The method of claim 5, wherein the tumor cells are ovarian tumor cells.

7. A method for identifying therapeutic agents for treating ovarian cancer cells, comprising introducing a candidate therapeutic agent, into an athymic Nu/Nu mouse, having ovarian carcinoma cells expressing SEAP implanted intraperitoneally.

8. The method of claim 7, wherein the ovarian carcinoma expressing SEAP is OCC1.

9. A method for monitoring tumor burden in a subject, comprising quantifying the amount of heat stable SEAP in a blood sample from an immunocompromised animal having tumor cells expressing heat stable SEAP, implanted intraperitoneally after treating said animal with the composition of claim 1.

10. The method of claim 9, wherein the tumor cells are ovarian cancer cells.

11. A pharmaceutical composition, comprising a suitable carrier, and an inhibitor of a PI3K signal transduction pathway, and an antagonist of lysophosphatidic acid (LPA).

12. The composition of claim 11, wherein the inhibitor of the PI3K signal transduction pathway is selected from at least one agent of the group consisting of LY294001, Wortmannin, PD098059 and U0126, and combinations of these agents, and the LPA antagonist is selected from the group consisting of the agents LPA 10:0, LPA 14:0, or LXR LPA.

13. The composition of claim 12, further comprising Triptolide.

14. A pharmaceutical composition, comprising a suitable carrier, and an inhibitor of a PI3K signal transduction pathway comprising at least one agent selected from the group consisting of PD09059, U016, LY294002, and/or Wortmannin, and Triptolide.

15. A method for monitoring tumor burden in a subject, comprising quantifying the amount of heat stable SEAP in a blood sample from an immunocompromised animal having tumor cells expressing heat stable SEAP, implanted intraperitoneally after treating said animal with the composition of claim 1, and the tumor cells are ovarian cancer cells.

16. A method for killing tumor cells, comprising administering to a subject a therapeutically effective amount of the pharmaceutical composition of claim 1, sufficient to kill the tumor cells.

17. A method for inhibiting tumor growth, comprising administering to a subject, a therapeutically effective amount of the pharmaceutical composition of claim 1, to inhibit tumor growth.

18. A method for inhibiting tumor growth, comprising administering to a subject, a therapeutically effective amount of the pharmaceutical composition of claim 1, in combination with a hormone regimen or radiotherapy.
